SEQUENCE LISTING (57 sequences)

SEQ ID NO: 1 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - protein, 415 aa, Artificial Sequence
  FIX variants
YNSGKLEEFVQGNLERECMEEKCSFEEAREVFENTERTTEFWKQYVDGDQCESNPCLNGGSCKDDINSYECWCPFGFEGKNCELXXXCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLXSTKFTIYNNMFCAGFHEGGRDSCQGDSGGPHVTEVEGTSFLTGIISWGEECAMKGKYGIYTKVSRYVNWIKXKTKLT

SEQ ID NO: 3 - DNA, 35 bp, Artificial Sequence
  FIXF1 5′ primer
atcataagcttgccaccatgcagcgcgtgaacatg

SEQ ID NO: 4 - DNA, 33 bp, Artificial Sequence
  FIXR3 3′ primer
atcataagcttgattagttagtgagaggccctg

SEQ ID NO: 5 - DNA, 47 bp, Artificial Sequence
  V86A primer
aggaaagaactgtgaattagatgccacatgtaacattaagaatggca

SEQ ID NO: 6 - DNA, 47 bp, Artificial Sequence
  V86A primer
tgccattcttaatgttacatgtggcatctaattcacagttctttcct

SEQ ID NO: 7 - DNA, 47 bp, Artificial Sequence
  V86P primer
ggaaagaactgtgaattagatcccacatgtaacattaagaatggcag

SEQ ID NO: 8 - DNA, 47 bp, Artificial Sequence
  V86P primer
ctgccattcttaatgttacatgtgggatctaattcacagttctttcc

SEQ ID NO: 9 - DNA, 47 bp, Artificial Sequence
  V86E primer
ggaaagaactgtgaattagatgagacctgtaacattaagaatggcag

SEQ ID NO: 10 - DNA, 47 bp, Artificial Sequence
  V86E primer
ctgccattcttaatgttacaggtctcatctaattcacagttctttcc

SEQ ID NO: 11 - DNA, 47 bp, Artificial Sequence
  V86S primer
ggaaagaactgtgaattagatagcacctgtaacattaagaatggcag

SEQ ID NO: 12 - DNA, 47 bp, Artificial Sequence
  V86S primer
ctgccattcttaatgttacaggtgctatctaattcacagttctttcc

SEQ ID NO: 13 - DNA, 47 bp, Artificial Sequence
  V86I primer
ggaaagaactgtgaattagatatcacctgtaacattaagaatggcag

SEQ ID NO: 14 - DNA, 47 bp, Artificial Sequence
  V86I primer
ctgccattcttaatgttacaggtgatatctaattcacagttctttcc

SEQ ID NO: 15 - DNA, 47 bp, Artificial Sequence
  V86R primer
ggaaagaactgtgaattagatagaacctgtaacattaagaatggcag

SEQ ID NO: 16 - DNA, 47 bp, Artificial Sequence
  V86R primer
ctgccattcttaatgttacaggttctatctaattcacagttctttcc

SEQ ID NO: 17 - DNA, 47 bp, Artificial Sequence
  V86Q primer
ggaaagaactgtgaattagatcagacatgtaacattaagaatggcag

SEQ ID NO: 18 - DNA, 47 bp, Artificial Sequence
  V86Q primer
ctgccattcttaatgttacatgtctgatctaattcacagttctttcc

SEQ ID NO: 19 - DNA, 47 bp, Artificial Sequence
  V86T primer
ggaaagaactgtgaattagataccacctgtaacattaagaatggcag

SEQ ID NO: 20 - DNA, 47 bp, Artificial Sequence
  V86T primer
ctgccattcttaatgttacaggtggtatctaattcacagttctttcc

SEQ ID NO: 21 - DNA, 47 bp, Artificial Sequence
  V86D primer
ggaaagaactgtgaattagatgacacctgtaacattaagaatggcag

SEQ ID NO: 22 - DNA, 47 bp, Artificial Sequence
  V86D primer
ctgccattcttaatgttacaggtgtcatctaattcacagttctttcc

SEQ ID NO: 23 - DNA, 47 bp, Artificial Sequence
  V86H primer
ggaaagaactgtgaattagatcacacctgtaacattaagaatggcag

SEQ ID NO: 24 - DNA, 47 bp, Artificial Sequence
  V86H primer
ctgccattcttaatgttacaggtgtgatctaattcacagttctttcc

SEQ ID NO: 25 - DNA, 47 bp, Artificial Sequence
  V86N primer
ggaaagaactgtgaattagataacacctgtaacattaagaatggcag

SEQ ID NO: 26 - DNA, 47 bp, Artificial Sequence
  V86N primer
ctgccattcttaatgttacaggtgttatctaattcacagttctttcc

SEQ ID NO: 27 - DNA, 47 bp, Artificial Sequence
  V86L primer
ggaaagaactgtgaattagatctgacatgtaacattaagaatggcag

SEQ ID NO: 28 - DNA, 47 bp, Artificial Sequence
  V86L primer
ctgccattcttaatgttacatgtcagatctaattcacagttctttcc

SEQ ID NO: 29 - DNA, 47 bp, Artificial Sequence
  V86M primer
ggaaagaactgtgaattagatatgacctgtaacattaagaatggcag

SEQ ID NO: 30 - DNA, 47 bp, Artificial Sequence
  V86M primer
ctgccattcttaatgttacaggtcatatctaattcacagttctttcc

SEQ ID NO: 31 - DNA, 47 bp, Artificial Sequence
  V86Y primer
ggaaagaactgtgaattagattacacctgtaacattaagaatggcag

SEQ ID NO: 32 - DNA, 47 bp, Artificial Sequence
  V86Y primer
ctgccattcttaatgttacaggtgtaatctaattcacagttctttcc

SEQ ID NO: 33 - DNA, 47 bp, Artificial Sequence
  V86K primer
ggaaagaactgtgaattagataagacatgtaacattaagaatggcag

SEQ ID NO: 34 - DNA, 47 bp, Artificial Sequence
  V86K primer
ctgccattcttaatgttacatgtcttatctaattcacagttctttcc

SEQ ID NO: 35 - DNA, 47 bp, Artificial Sequence
  V86F primer
ggaaagaactgtgaattagatttcacctgtaacattaagaatggcag

SEQ ID NO: 36 - DNA, 47 bp, Artificial Sequence
  V86F primer
ctgccattcttaatgttacaggtgaaatctaattcacagttctttcc

SEQ ID NO: 37 - DNA, 47 bp, Artificial Sequence
  V86C primer
ggaaagaactgtgaattagattgcacctgtaacattaagaatggcag

SEQ ID NO: 38 - DNA, 46 bp, Artificial Sequence
  V86C primer
ctgccattcttaatgttacaggtgcaatctaattcacagttctttc

SEQ ID NO: 39 - DNA, 47 bp, Artificial Sequence
  V86W primer
ggaaagaactgtgaattagattggacctgtaacattaagaatggcag

SEQ ID NO: 40 - DNA, 47 bp, Artificial Sequence
  V86W primer
ctgccattcttaatgttacaggtccaatctaattcacagttctttcc

SEQ ID NO: 41 - DNA, 55 bp, Artificial Sequence
  V86G primer
gaaggaaagaactgtgaattagatggcacctgtaacattaagaatggcagatgcg

SEQ ID NO: 42 - DNA, 55 bp, Artificial Sequence
  V86G primer
cgcatctgccattcttaatgttacaggtgccatctaattcacagttctttccttc

SEQ ID NO: 43 - DNA, 51 bp, Artificial Sequence
  E410N primer
tcccggtatgtcaactggattaagaacaaaacaaagctcacttaatgaaag

SEQ ID NO: 44 - DNA, 51 bp, Artificial Sequence
  E410N primer
ctttcattaagtgagctttgttttgttcttaatccagttgacataccggga

SEQ ID NO: 45 - DNA, 51 bp, Artificial Sequence
  E410Q primer
tcccggtatgtcaactggattaagcagaaaacaaagctcacttaatgaaag

SEQ ID NO: 46 - DNA, 51 bp, Artificial Sequence
  E410Q primer
ctttcattaagtgagctttgttttctgcttaatccagttgacataccggga

SEQ ID NO: 47 - DNA, 52 bp, Artificial
  Primer
ctgtttttcctgatgtggactacgtagcctctactgaagctgaaaccattct

SEQ ID NO: 48 - DNA, 44 bp, Artificial
  Primer
gaagctgaaaccattctagatgccatcactcaaagcacccaatc

SEQ ID NO: 49 - DNA, 45 bp, Artificial
  Primer
cttgttgaccgagccacatgccttgcatctacaaagttcaccatc

SEQ ID NO: 50 - DNA, 45 bp, Artificial
  Primer
cttgttgaccgagccacatgccttctgtctacaaagttcaccatc

SEQ ID NO: 51 - DNA, 39 bp, Artificial
  Primer
gaccgagccacatgccttgtgtctacaaagttcaccatc

SEQ ID NO: 52 - DNA, 48 bp, Artificial
  Primer
gttgaccgagccacatgccttatctctacaaagttcaccatctataac

SEQ ID NO: 53 - DNA, 48 bp, Artificial
  Primer
gttgaccgagccacatgccttttctctacaaagttcaccatctataac

SEQ ID NO: 54 - DNA, 45 bp, Artificial
  Primer
cttgttgaccgagccacatgcctttggtctacaaagttcaccatc

SEQ ID NO: 55 - DNA, 47 bp, Artificial
  Primer
cacttgttgaccgagccacatgccttatgtctacaaagttcaccatc

SEQ ID NO: 56 - DNA, 45 bp, Artificial
  Primer
cttgttgaccgagccacatgccttagctctacaaagttcaccatc

SEQ ID NO: 57 - DNA, 42 bp, Artificial
  Primer
gttgaccgagccacatgccttacctctacaaagttcaccatc

(19) United States
(12) Patent Application Publication
(10) Pub. No.: US 2012/0164130 A1
(43) Pub. Date: Jun. 28, 2012

(54) Modified Factor IX Polypeptides and Uses Thereof

(75) Inventors: Alan Brooks, Clayton, CA (US); Chandra Patel, Foster City, CA (US); Xiaoqiao Jiang, Concord, CA (US); Uwe Gritzan, Cologne (DE); Heiner Apeler, Wuppertal (DE); Jun Wang, Shanghai (CN)

(73) Assignee: BAYER HEALTHCARE LLC, Tarrytown, NY (US)

(21) Appl. No.: 13/388,288

(22) PCT Filed: Aug. 2, 2010
(86) PCT No.: PCT/US2010/044177

§ 371 Date: Mar. 14, 2012

Related U.S. Application Data
(60) Provisional application No. 61/230,551, filed on Jul. 31, 2009.

(51) Int. Cl. A61K 38/48; C12N 15/57; C12N 9/96; C07K 17/10; A61P 7/04; C12N 5/10; C12N 9/48; C07K 17/02

(52) U.S. Cl. 424/94.64; 435/212; 536/23.2; 435/365; 435/352; 435/369; 435/353; 435/358; 435/188

(57) ABSTRACT

The invention relates to modified Factor IX polypeptides such as Factor IX polypeptides with one or more amino acid substitutions. The invention also relates to methods of making modified Factor IX polypeptides, and methods of using modified Factor IX polypeptides, for example, to treat patients afflicted with hemophilia B.

### REFERENCE TO RELATED APPLICATIONS

This application claims priority to U.S. Provisional Application Ser. No. 61/230,551 filed on Jul. 31, 2009 and is hereby incorporated by reference for all purposes.

### FIELD

This application relates to modified Factor IX polypeptides, for example, Factor IX polypeptides that exhibit increased specific activity and polymer conjugated Factor IX polypeptides. This application also relates to methods of making modified Factor IX polypeptides and conjugates thereof, and methods of using modified Factor IX polypeptides, for example, to treat patients afflicted with hemophilia B.

### BACKGROUND

Hemophilia B effects one out of 34,500 males and is caused by various genetic defects in the gene encoding coagulation Factor IX (FIX) that result in either low or undetectable FIX protein in the blood (Kurachi, et al., Hematol. Oncol. Clin. North Am. 6:991-997, 1992; Lillicrap, Haemophilia 4:350-357, 1998). Insufficient levels of FIX lead to defective coagulation and symptoms that result from uncontrolled bleeding. Hemophilia B is treated effectively by the intravenous infusion of either plasma-derived or recombinant FIX protein either to stop bleeds that have already initiated or to prevent bleeding from occurring (prophylaxis) (Dargaud, et al., Expert Opin. Biol. Ther. 7:651-663; Giangrande, Expert Opin. Pharmacother. 6:1517-1524, 2005). Effective prophylaxis requires maintaining a minimum trough level of FIX of about 1% of normal levels (Giangrande, Expert Opin. Pharmacother. 6:1517-1524, 2005). Because of the approximately 18 to 24 hour half-life of native FIX (either plasma-derived or recombinant), FIX levels drop to less than 1% of normal levels within 3 to 4 days following bolus injection which necessitates repeat injection on average every three days to achieve effective prophylaxsis (Giangrande, Expert Opin. Pharmacother. 6:1517-1524, 2005). Such frequent intravenous injection is problematic for patients and is a hurdle for achieving effective prophylaxsis (Petrini, Haemophilia 13 Suppl 2:16-22, 2007), especially in children. A FIX protein with an increased specific activity has the potential to increase the duration of protection and thus, be of significant medical benefit.

### SUMMARY

The application provides FIX polypeptides (also referred to as modified FIX polypeptides, FIX muteins, or FIX variants) comprising amino acid sequences that have been modified to improve the specific activity of FIX. In some embodiments, the one or more amino acid substitutions have been introduced. In some embodiments, the polypeptides have coagulation activity. In some embodiments, the modified FIX polypeptides may comprise at least one substitution at amino acid residues 85, 86, 87, 338, and 410.

The modified FIX polypeptides may be generated by the introduction of one or more amino acid substitutions, for example, by substitution with any amino acid. Exemplary embodiments include FIX polypeptides comprising one or more substitutions such as, but not limited to:

(a) D85F; D85G; D85H; D85I; D85M; D85N; D85R; D85S; D85W; D85Y; V86A; V86D; V86E; V86G; V86H; V86I; V86L; V86M; V86N; V86P; V86Q; V86R; V86S; V86T; T87F; T87I; T87K; T87M; T87R; T87V; T87W; R338A; R338F; R338I; R338L; R338M; R338S; R338T; R338V; R338W; E410N; E410Q;
(b) D85W and T87R; D85F and T87I; D85W and T87W; D85R and T85R; D85I and T87R; D85Y and T87F; D85I and T87M; D85F and T87R; D85F and T87V; D85R and T87K; D85H and T87I; D85I and T87I; D85Y and T87K; D85S and T87R; D85Y and T87R; D85G and T87K; D85H and T87W; D85H and T87K; D85F and T87K; D85H and T87V; D85M and T87I; D85H and T87M; R338A and E410N; R338A and E410Q;
(c) D85W, V86A, and T87R; D85F, V86A, and T87I; D85W, V86A, and T87W; D85R, V86A, and T85R; D85I, V86A, and T87R; D85Y, V86A, and T87F; D85I, V86A, and T87M; D85F, V86A, and T87R; D85F, V86A, and T87V; D85R, V86A, and T87K; D85H, V86A, and T87I; D85I, V86A, and T87I; D85Y, V86A, and T87K; D85S, V86A, and T87R; D85Y, V86A, and T87R; D85G, V86A, and T87K; D85H, V86A, and T87W; D85H, V86A, and T87K; D85F, V86A, and T87K; D85H, V86A, and T87V; D85M, V86A, and T87I; D85H, V86A, and T87M;
(d) D85W, V86A, T87R, and R338A; D85F, V86A, T87I, and R338A; D85W, V86A, T87W, and R338A; D85R, V86A, T85R, and R338A; D85I, V86A, T87R, and R338A; D85Y, V86A, T87F, and R338A; D85I, V86A, T87M, and R338A; D85F, V86A, T87R, and R338A; D85F, V86A, T87V, and R338A; D85R, V86A, T87K, and R338A; D85H, V86A, T87I, and R338A; D85I, V86A, T87I, and R338A; D85Y, V86A, T87K, and R338A; D85S, V86A, T87R, and R338A; D85Y, V86A, T87R, and R338A; D85G, V86A, T87K, and R338A; D85H, V86A, T87W, and R338A; D85H, V86A, T87K, and R338A; D85F, V86A, T87K, and R338A; D85H, V86A, T87V, and R338A; D85M, V86A, T87I, and R338A; D85H, V86A, T87M, and R338A;
(e) D85W, V86A, T87R, R338A, and E410N; D85F, V86A, T87I, R338A, and E410N; D85W, V86A, T87W, R338A, and E410N; D85R, V86A, T85R, R338A, and E410N; D85I, V86A, T87R, R338A, and E410N; D85Y, V86A, T87F, R338A, and E410N; D85I, V86A, T87M, R338A, and E410N; D85F, V86A, T87R, R338A, and E410N; D85F, V86A, T87V, R338A, and E410N; D85R, V86A, T87K, R338A, and E410N; D85H, V86A, T87I, R338A, and E410N; D85I, V86A, T87I, R338A, and E410N; D85Y, V86A, T87K, R338A, and E410N; D85S, V86A, T87R, R338A, and E410N; D85Y, V86A, T87R, R338A, and E410N; D85G, V86A, T87K, R338A, and E410N; D85H, V86A, T87W, R338A, and E410N; D85H, V86A, T87K, R338A, and E410N; D85F, V86A, T87K, R338A, and E410N; D85H, V86A, T87V, R338A, and E410N; D85M, V86A, T87I, R338A, and E410N; D85H, V86A, T87M, R338A, and E410N; D85W, V86A, T87R, R338A, and E410Q; D85F, V86A, T87I, R338A, and E410Q; D85W, V86A, T87W, R338A, and E410Q; D85R, V86A, T85R, R338A, and E410Q; D85I, V86A, T87R, R338A, and E410Q; D85Y, V86A, T87F, R338A, and E410Q; D85I, V86A, T87M, R338A, and E410Q; D85F, V86A, T87R, R338A, and E410Q; D85F, V86A, T87V, R338A, and E410Q; D85R, V86A, T87K, R338A, and E410Q; D85H, V86A, T87I, R338A, and E410Q; D85I, V86A, T87I, R338A, and E410Q; D85Y, V86A, T87K, R338A, and E410Q; D85S, V86A, T87R, R338A, and E410Q; D85Y, V86A, T87R, R338A, and E410Q; D85G, V86A, T87K, R338A, and E410Q; D85H, V86A, T87W, R338A, and E410Q; D85H, V86A, T87K, R338A, and E410Q; D85F, V86A, T87K, R338A, and E410Q; D85H, V86A, T87V, R338A, and E410Q; D85M, V86A, T87I, R338A, and E410Q; D85H, V86A, T87M, R338A, and E410Q; and any combination thereof.

The application also provides FIX polypeptide conjugates comprising amino acid sequences that have been modified to improve the specific activity of FIX and one or more polymer moieties covalently attached to the FIX polypeptide. In some embodiments, the polymer moieties are covalently attached to sugar moieties on the FIX polypeptide, wherein the sugar moieties are naturally attached to the peptide during expression in mammalian cells.

The application also provides pharmaceutical preparations comprising modified FIX polypeptides and a pharmaceutically acceptable carrier.

The application also provides methods for treating hemophilia B comprising administering to a subject in need thereof a therapeutically effective amount of the pharmaceutical preparations described herein.

The application also provides DNA sequences encoding modified polypeptides, as well as eukaryotic host cells transfected with the DNA sequences.

The application also provides methods for producing modified FIX polypeptides comprising (i) modifying the amino acid sequence of the polypeptide by introducing one or more amino acid substitutions; (ii) expressing the polypeptide in, for example, a mammalian cell line; and (iii) purifying the polypeptide.

The application also provides a conjugate comprising a) a Factor IX polypeptide comprising an amino acid sequence that has been modified by introducing one or more amino acid substitutions, wherein at least one amino acid substitution is at residue 338; b) one or more sugar moieties attached to said one or more glycosylation sites; and c) one or more polymer moieties covalently attached to one or more sugar moieties.

The application also provides a method for improving conjugation of a polymer moiety to a polypeptide comprising: a) providing a polypeptide having one or more glycosylation sites, wherein the glycosylation site comprises one or more sialic acids; b) oxidizing said sialic acids of said polypeptide; c) providing a catalyst; and d) covalently attaching a polymer moiety comprising an amino-oxy functional group to said oxidized sialic acids.

### DESCRIPTION OF THE DRAWINGS

FIG. 1 depicts a graph showing dose normalized pharmacokinetic profile of glycoPEGylated FIX-R338A, FIX-R338A and recombinant wild type FIX in normal rats.

FIG. 2 depicts a graph showing a pharmacokinetic profile of glycoPEGylated FIX-R338A, FIX-R338A and rFIX in Hemophilia B mice.

FIG. 3 depicts a graph showing FIX activity in the plasma of Hemophilia B mice following intravenous injection of rFIX, FIX-R338A or glycoPEGylated FIX-R338A.

FIG. 4 shows a time course analysis by SDS-PAGE of the PEGylation reaction with and without a catalyst.

### DESCRIPTION OF THE INVENTION

The present application provides FIX polypeptides that include one or more amino acid substitutions. For example, the modified FIX polypeptides may comprise at least one substitution at amino acid residues 85, 86, 87, 338, and 410. The modified FIX polypeptides may have an increased specific activity that would provide, for example, an extended time of protection against bleeding in hemophilia B patients. The modified FIX polypeptides would enable hemophilia B patients to achieve protection against bleeding with fewer injections of FIX than is possible with the currently available therapy of wild type FIX protein.

Activated Factor VII (FVII) initiates the normal hemostatic process by forming a complex with tissue factor (TF), exposed as a result of injury to the vessel wall. The complex subsequently activates FIX; the active form referred to as FIXa. The activation peptide of FIX is removed by proteolytic cleavage at two sites by either Factor XIa (FXIa) or the tissue factor (TF)/Factor VIIa complex to generate the catalytically active molecule, Factor IXa (FIXa). FIXa and Factor VIIIa (FVIIIa) convert FX to Factor Xa (FXa), which in turn converts prothrombin to thrombin. Thrombin then converts fibrinogen to fibrin resulting in formation of a fibrin clot.

As wild-type FIX has numerous post-translational modifications some of which have been suggested to play a role in the in vivo pharmacokinetic profile. Once produced, FIX should retain enzymatic activity and interact with FVIII, FXI, and FX in order to be an effective treatment for hemophilia B. The introduction of substituted amino acids should not perturb these interactions and function. The application provides, in part, modifications to FIX which are likely to result in an increased specific activity with minimal perturbation of function. Alterations that enhance the specific activity of FIX may compensate for potential loss of coagulation activity and also potentially prolong the efficacy of modified molecules by conferring efficacy at lower levels of protein.

### Modified FIX Polypeptides

The application provides FIX polypeptides comprising one or more amino acid substitutions, that is, modified FIX polypeptides. “Factor IX” as used herein refers to a FIX protein that is a member of the intrinsic coagulation pathway and is essential to blood coagulation. It is to be understood that this definition includes native as well as recombinant forms of the FIX protein. Unless otherwise specified or indicated, as used herein FIX means any functional human FIX protein molecule in its normal role in coagulation, including any fragment, analogue, variant, and derivative thereof. The terms “fragment,” “derivative,” “analogue,” “mutein,” and “variant,” when referring to the polypeptides of the application, means fragments, derivatives, analogues, muteins, and variants of the polypeptides which retain substantially the same biological function or activity.

Non-limiting examples of FIX polypeptides include FIX, FIXa, and truncated versions of FIX having FIX activity. Biologically active fragments, deletion variants, substitution variants, or addition variants of any of the foregoing that maintain at least some degree of FIX activity can also serve as a FIX polypeptide. In some embodiments, the FIX polypeptides may comprise an amino acid sequence at least about 70, 80, 90, or 95% identical to SEQ ID NO: 1. In some embodiments, the modified FIX polypeptides are biologically active. Biological activity can be determined, for example, by coagulation assays described herein.

Modified FIX polypeptides may contain conservative substitutions of amino acids. A conservative substitution is recognized in the art as a substitution of one amino acid for another amino acid that has similar properties and include, for example, the changes of alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. In some embodiments, the FIX polypeptides of SEQ ID NO: 1 comprise from 1-30, from 1-20, or from 1-10 conservative amino acid substitutions.

The single letter abbreviation for a particular amino acid, its corresponding amino acid, and three letter abbreviation are as follows: A, alanine (Ala); C, cysteine (Cys); D, aspartic acid (Asp); E, glutamic acid (Glu); F, phenylalanine (Phe); G, glycine (Gly); H, histidine (His); I, isoleucine (Ile); K, lysine (Lys); L, leucine (Leu); M, methionine (Met); N, asparagine (Asn); P, proline (Pro); Q, glutamine (Gln); R, arginine (Arg); S, serine (Ser); T, threonine (Thr); V, valine (Val); W, tryptophan (Trp); Y, tyrosine (Tyr); and norleucine (Nle).

The modified FIX polypeptides may also be glycosylated. Glycosylation of polypeptides is typically either N-linked or O-linked N-linked refers to the attachment of a carbohydrate moiety to the side chain of an asparagine residue. The tripeptide sequences Asn-X-Ser and Asn-X-Thr, where X is any amino acid except proline, are the recognition sequences for enzymatic attachment of the carbohydrate moiety to the Asn side chain. Thus, the presence of either of these tripeptide sequences in a polypeptide creates a potential N-linked glycosylation site. An exemplary N-linked glycosylation site may be represented as follows X1-Asn-X2-X3-X4; where X1 is optionally Asp, Val, Glu, Gly, or Ile; X2 is any amino acid except Pro; X3 is Ser or Thr; and X4 is optionally Val, Glu, Gly, Gln, or Ile. Addition of N-linked glycosylation sites to a FIX polypeptide is accomplished by altering the amino acid sequence such that one or more of the above-described tripeptide sequences is introduced.

O-linked glycosylation refers to the attachment of one of the sugars N-aceytlgalactosamine, galactose, or xylose to a hydroxyamino acid, most commonly to serine or threonine, although attachment to 5-hydroxyproline or 5-hydroxylysine is also possible. Addition of O-linked glycosylation sites to a FIX polypeptide may be accomplished by altering the amino acid sequence such that one or more Ser or Thr residues are introduced.

Glycosylation sites may be introduced, for example, by deleting one or more amino acid residues, substituting one or more endogenous FIX amino acid residues with another amino acid(s), or adding one or more amino acid residues. The addition of an amino acid residue may be either between two existing amino acid residues or at the N- or C-terminal end of the native FIX molecule.

The terminology for amino acid substitutions used is as follows. The first letter represents the amino acid residue naturally present at a position of human FIX. The following number represents the position in the mature human FIX amino acid sequence (SEQ ID NO:1). The second letter represent the different amino acid substituting for (replacing/substituting) the natural amino acid. As an example, V86A denotes that the Val residue at position 86 of SEQ ID NO: 1 has been replaced with an Ala residue.

The FIX residue number system used herein refers to that of the mature human FIX protein in which residue 1 represents the first amino acid of the mature FIX polypeptide following removal of both the signal sequence and the propeptide. Native or wild type FIX is the full length mature human FIX molecule as shown in SEQ ID NO: 1.

It may be desirable to compare the properties of the modified FIX polypeptides having one or more amino acid substitutions to a control polypeptide. Properties for comparison include, for example, solubility, activity, plasma half-life, and binding properties. It is within the purview of one skilled in the art to select the most appropriate control polypeptide for comparison. In some embodiments, the control polypeptide may be identical to the modified polypeptide except for the one or more amino acid substitutions. Exemplary polypeptides include wild-type FIX polypeptide and FIX polypeptides comprising one or more activating substitutions, such as R338A and/or V86A.

One aspect of the application provides modified FIX polypeptides having increased specific activity as compared to a control polypeptide. Enhanced specific activity may be desirable to reduce the frequency of dosing that is required to achieve therapeutic effectiveness. Accordingly, in certain embodiments, the FIX polypeptides have a specific activity increased by about 20, 30, 40, 60, 80, 100, 150, 200, 300, 400, 500, 600, 700, 800, 900, or 1000% relative to a control protein.

The term “half-life,” as used herein in the context of administering a polypeptide drug to a patient, is defined as the time required for plasma concentration of a drug in a patient to be reduced by one half. Methods for pharmacokinetic analysis and determination of half-life and in vivo stability will be familiar to those skilled in the art. Details may be found in Kenneth, et al., Chemical Stability of Pharmaceuticals: A Handbook for Pharmacists and in Peters, et al., Pharmacokinetic analysis: A Practical Approach (1996). Reference is also made to “Pharmacokinetics,” M Gibaldi & D Perron, published by Marcel Dekker, 2nd Rev. edition (1982), which describes pharmacokinetic parameters such as t-alpha and t-beta half lives and area under the curve (AUC).

The activity of modified FIX polypeptides may be described either as an absolute value, such as in units, or as a percentage of the activity of a control polypeptide. FIX specific activity may be defined as the ability to function in the coagulation cascade, induce the formation of FXa via interaction with FVIIIa on an activated platelet, or support the formation of a blood clot. The activity may be assessed in vitro by techniques such as clot analysis, as described in, for example, McCarthy, et al., (Thromb. Haemost. 87:824-830, 2002), and other techniques known to those skilled in the art. The activity may also be assessed in vivo using one of the several animal lines that have been intentionally bred with a genetic mutation for hemophilia B such that an animal produced from such a line is deficient for FIX. Such lines are available from a variety of sources such as, without limitation, the Division of Laboratories and Research, New York Department of Public Health, Albany, N.Y. and the Department of Pathology, University of North Carolina, Chapel Hill, N.C. Both of these sources, for example, provide canines suffering from canine hemophilia B. Alternatively, mice deficient in FIX are also available (Sabatino, et al., Blood 104:2767-2774, 2005). In order to test for FIX activity, a test polypeptide is injected into the diseased animal, a small cut made and bleeding time compared to a healthy control.

Human wild-type FIX has a specific activity of around 200 units per mg. One unit of FIX has been defined as the amount of FIX present in one millilitre of normal (pooled) human plasma (corresponding to a FIX level of 100%). In some embodiments, the modified FIX polypeptides may have a specific activity of at least about 200 units, 300 units, 400 units, 500 units, or more per mg of FIX polypeptide. In some embodiments, the modified FIX polypeptides may have a specific activity of at least about 500 units, 600 units, 700 units, 750 units or more per mg of FIX polypeptide. In some embodiments, the specific activity of FIX may be measured using the APTT or activated partial thromboplastin time assay (described by, for example, Proctor, et al., Am. J. Clin. Pathol. 36:212, 1961).

When expressed in cells, such as liver or kidney cells, FIX polypeptide may be synthesized by the cellular machinery, undergoes posttranslational modification, and is then secreted by the cells into the extracellular milieu. The amount of FIX polypeptide secreted from cells is therefore dependent on both processes of protein translation and extracellular secretion. In some embodiments, the modified FIX polypeptides may be secreted in an amount that is not reduced more than about 10, 20, 30, 40, 50, 60, 70, or 80% relative to the amount secreted of a control protein. For example, a modified FIX polypeptide may be secreted in an amount that is not reduced more than about 80% relative to a control FIX polypeptide, if the modified polypeptide is secreted in an amount of at least about 20% as compared to the control. The amount of FIX polypeptide secreted may be measured, for example, by determining the protein levels in the extracellular medium using any art-known method. Traditional methodologies for protein quantification include 2-D gel electrophoresis, mass spectrometry, and antibody binding. Exemplary methods for assaying protein levels in a biological sample include antibody-based techniques, such as immunoblotting (western blotting), immunohistological assay, enzyme linked immunosorbent assay (ELISA), or radioimmunoassay (RIA).

In some embodiments, the modified FIX polypeptides interact with at least one of FVIII, FXI, or FX at a level not reduced more than about 40, 50, 60, 70, or 80% relative to the interaction of a control protein with at least one of FVIII, FXI, or FX. For example, a modified FIX polypeptide may interact with at least one of FVIII, FXI, or FX at a level not reduced more than about 80% relative to a control FIX polypeptide, if the modified polypeptide interacts with at least one of FVIII, FXI, or FX at a level of at least about 20% as compared to the control. The binding of FIX to other members of the coagulation cascade can be determined by any method known to one skilled in the art, including for example, the methods described in Chang, et al., (J. Biol. Chem. 273:12089-12094, 1998).

The application provides, in part, FIX polypeptides comprising one or more amino acid substitutions. In some embodiments, FIX polypeptides are provided comprising one or more substitutions selected from D85F; D85G; D85H; D85I; D85M; D85N; D85R; D85S; D85W; D85Y; V86A; V86D; V86E; V86G; V86H; V86I; V86L; V86M; V86N; V86P; V86Q; V86R; V86S; V86T; T87F; T87I; T87K; T87M; T87R; T87V; T87W; R338A; R338F; R338I; R338L; R338M; R338S; R338T; R338V; R338W; E410N; E410Q; or any combination thereof.

In some embodiments, FIX polypeptides are provided comprising one or more substitutions selected from D85W and T87R; D85F and T87I; D85W and T87W; D85R and T85R; D85I and T87R; D85Y and T87F; D85I and T87M; D85F and T87R; D85F and T87V; D85R and T87K; D85H and T87I; D85I and T87I; D85Y and T87K; D85S and T87R; D85Y and T87R; D85G and T87K; D85H and T87W; D85H and T87K; D85F and T87K; D85H and T87V; D85M and T87I; D85H and T87M; R338A and E410N; R338A and E410Q; or any combination thereof.

In some embodiments, FIX polypeptides are provided comprising one or more substitutions selected from D85W, V86A, and T87R; D85F, V86A, and T87I; D85W, V86A, and T87W; D85R, V86A, and T85R; D85I, V86A, and T87R; D85Y, V86A, and T87F; D85I, V86A, and T87M; D85F, V86A, and T87R; D85F, V86A, and T87V; D85R, V86A, and T87K; D85H, V86A, and T87I; D85I, V86A, and T87I; D85Y, V86A, and T87K; D85S, V86A, and T87R; D85Y, V86A, and T87R; D85G, V86A, and T87K; D85H, V86A, and T87W; D85H, V86A, and T87K; D85F, V86A, and T87K; D85H, V86A, and T87V; D85M, V86A, and T87I; D85H, V86A, and T87M; or any combination thereof.

In some embodiments, FIX polypeptides are provided comprising one or more substitutions selected from D85W, V86A, T87R, and R338A; D85F, V86A, T87I, and R338A; D85W, V86A, T87W, and R338A; D85R, V86A, T85R, and R338A; D85I, V86A, T87R, and R338A; D85Y, V86A, T87F, and R338A; D85I, V86A, T87M, and R338A; D85F, V86A, T87R, and R338A; D85F, V86A, T87V, and R338A; D85R, V86A, T87K, and R338A; D85H, V86A, T87I, and R338A; D85I, V86A, T87I, and R338A; D85Y, V86A, T87K, and R338A; D85S, V86A, T87R, and R338A; D85Y, V86A, T87R, and R338A; D85G, V86A, T87K, and R338A; D85H, V86A, T87W, and R338A; D85H, V86A, T87K, and R338A; D85F, V86A, T87K, and R338A; D85H, V86A, T87V, and R338A; D85M, V86A, T87I, and R338A; D85H, V86A, T87M, and R338A; or any combination thereof.

In some embodiments, FIX polypeptides are provided comprising one or more substitutions selected from D85W, V86A, T87R, R338A, and E410N; D85F, V86A, T87I, R338A, and E410N; D85W, V86A, T87W, R338A, and E410N; D85R, V86A, T85R, R338A, and E410N; D85I, V86A, T87R, R338A, and E410N; D85Y, V86A, T87F, R338A, and E410N; D85I, V86A, T87M, R338A, and E410N; D85F, V86A, T87R, R338A, and E410N; D85F, V86A, T87V, R338A, and E410N; D85R, V86A, T87K, R338A, and E410N; D85H, V86A, T87I, R338A, and E410N; D85I, V86A, T87I, R338A, and E410N; D85Y, V86A, T87K, R338A, and E410N; D85S, V86A, T87R, R338A, and E410N; D85Y, V86A, T87R, R338A, and E410N; D85G, V86A, T87K, R338A, and E410N; D85H, V86A, T87W, R338A, and E410N; D85H, V86A, T87K, R338A, and E410N; D85F, V86A, T87K, R338A, and E410N; D85H, V86A, T87V, R338A, and E410N; D85M, V86A, T87I, R338A, and E410N; D85H, V86A, T87M, R338A, and E410N; D85W, V86A, T87R, R338A, and E410Q; D85F, V86A, T87I, R338A, and E410Q; D85W, V86A, T87W, R338A, and E410Q; D85R, V86A, T85R, R338A, and E410Q; D85I, V86A, T87R, R338A, and E410Q; D85Y, V86A, T87F, R338A, and E410Q; D85I, V86A, T87M, R338A, and E410Q; D85F, V86A, T87R, R338A, and E410Q; D85F, V86A, T87V, R338A, and E410Q; D85R, V86A, T87K, R338A, and E410Q; D85H, V86A, T87I, R338A, and E410Q; D85I, V86A, T87I, R338A, and E410Q; D85Y, V86A, T87K, R338A, and E410Q; D85S, V86A, T87R, R338A, and E410Q; D85Y, V86A, T87R, R338A, and E410Q; D85G, V86A, T87K, R338A, and E410Q; D85H, V86A, T87W, R338A, and E410Q; D85H, V86A, T87K, R338A, and E410Q; D85F, V86A, T87K, R338A, and E410Q; D85H, V86A, T87V, R338A, and E410Q; D85M, V86A, T87I, R338A, and E410Q; D85H, V86A, T87M, R338A, and E410Q; and any combination thereof.

In some embodiments, FIX polypeptides are provided comprising one or more substitutions selected from

(a) D85F; D85G; D85H; D85I; D85M; D85N; D85R; D85S; D85W; D85Y; V86A; V86D; V86E; V86G; V86H; V86I; V86L; V86M; V86N; V86P; V86Q; V86R; V86S; V86T; T87F; T87I; T87K; T87M; T87R; T87V; T87W; R338A; R338F; R338I; R338L; R338M; R338S; R338T; R338V; R338W; E410N; E410Q;
(b) D85W and T87R; D85F and T87I; D85W and T87W; D85R and T85R; D85I and T87R; D85Y and T87F; D85I and T87M; D85F and T87R; D85F and T87V; D85R and T87K; D85H and T87I; D85I and T87I; D85Y and T87K; D85S and T87R; D85Y and T87R; D85G and T87K; D85H and T87W; D85H and T87K; D85F and T87K; D85H and T87V; D85M and T87I; D85H and T87M;
(c) D85W, V86A, and T87R; D85F, V86A, and T871; D85W, V86A, and T87W; D85R, V86A, and T85R; D851, V86A, and T87R; D85Y, V86A, and T87F; D851, V86A, and T87M; D85F, V86A, and T87R; D85F, V86A, and T87V; D85R, V86A, and T87K; D85H, V86A, and T871; D851, V86A, and T871; D85Y, V86A, and T87K; D85S, V86A, and T87R; D85Y, V86A, and T87R; D85G, V86A, and T87K; D85H, V86A, and T87W; D85H, V86A, and T87K; D85F, V86A, and T87K; D85H, V86A, and T87V; D85M, V86A, and T871; D85H, V86A, and T87M; R338A and E410N; R338A and E410Q;
(d) D85W, V86A, T87R, and R338A; D85F, V86A, T871, and R338A; D85W, V86A, T87W, and R338A; D85R, V86A, T85R, and R338A; D851, V86A, T87R, and R338A; D85Y, V86A, T87F, and R338A; D851, V86A, T87M, and R338A; D85F, V86A, T87R, and R338A; D85F, V86A, T87V, and R338A; D85R, V86A, T87K, and R338A; D85H, V86A, T871, and R338A; D851, V86A, T871, and R338A; D85Y, V86A, T87K, and R338A; D85S, V86A, T87R, and R338A; D85Y, V86A, T87R, and R338A; D85G, V86A, T87K, and R338A; D85H, V86A, T87W, and R338A; D85H, V86A, T87K, and R338A; D85F, V86A, T87K, and R338A; D85H, V86A, T87V, and R338A; D85M, V86A, T871, and R338A; D85H, V86A, T87M, and R338A;
(e) D85W, V86A, T87R, R338A, and E410N; D85F, V86A, T871, R338A, and E410N; D85W, V86A, T87W, R338A, and E410N; D85R, V86A, T85R, R338A, and E410N; D851, V86A, T87R, R338A, and E410N; D85Y, V86A, T87F, R338A, and E410N; D851, V86A, T87M, R338A, and E410N; D85F, V86A, T87R, R338A, and E410N; D85F, V86A, T87V, R338A, and E410N; D85R, V86A, T87K, R338A, and E410N; D85H, V86A, T871, R338A, and E410N; D851, V86A, T871, R338A, and E410N; D85Y, V86A, T87K, R338A, and E410N; D85S, V86A, T87R, R338A, and E410N; D85Y, V86A, T87R, R338A, and E410N; D85G, V86A, T87K, R338A, and E410N; D85H, V86A, T87W, R338A, and E410N; D85H, V86A, T87K, R338A, and E410N; D85F, V86A, T87K, R338A, and E410N; D85H, V86A, T87V, R338A, and E410N; D85M, V86A, T871, R338A, and E410N; D85H, V86A, T87M, R338A, and E410N; D85W, V86A, T87R, R338A, and E410Q; D85F, V86A, T871, R338A, and E410Q; D85W, V86A, T87W, R338A, and E410Q; D85R, V86A, T85R, R338A, and E410Q; D851, V86A, T87R, R338A, and E410Q; D85Y, V86A, T87F, R338A, and E410Q; D851, V86A, T87M, R338A, and E410Q; D85F, V86A, T87R, R338A, and E410Q; D85F, V86A, T87V, R338A, and E410Q; D85R, V86A, T87K, R338A, and E410Q; D85H, V86A, T871, R338A, and E410Q; D851, V86A, T871, R338A, and E410Q; D85Y, V86A, T87K, R338A, and E410Q; D85S, V86A, T87R, R338A, and E410Q; D85Y, V86A, T87R, R338A, and E410Q; D85G, V86A, T87K, R338A, and E410Q; D85H, V86A, T87W, R338A, and E410Q; D85H, V86A, T87K, R338A, and E410Q; D85F, V86A, T87K, R338A, and E410Q; D85H, V86A, T87V, R338A, and E410Q; D85M, V86A, T871, R338A, and E410Q; D85H, V86A, T87M, R338A, and E410Q; and any combination thereof.

A further aspect of the application provides FIX polypeptides with increased specific activity. In some embodiments, the polypeptides may have a specific activity of at least about 200, 300, 400, 500, 600, 700, 800, 900, 1000, 1100, 1200, 1400, 1600, 1800, 2000, 4000, 6000, 8000, or more units per mg of polypeptide. The specific activity can be determined as previously described, such as, for example, using the APTT assay. These polypeptides are useful as therapeutic agents, particularly in patients afflicted with hemophilia B. These polypeptides may comprise further substitutions or modifications, such as the glycosylation sites described herein.

One aspect of the application provides modified Factor IX polypeptides comprising the following amino acid sequence:

(SEQ ID NO: 2)
YNSGKLEEFVQGNLERECMEEKCSFEEAREVFENTERTTEFWKQYVDGD
QCESNPCLNGGSCKDDINSYECWCPFGFEGKNCELX₈₅X₈₆X₈₇CNIKN
GRCEQFCKNSADNKVVCSCTEGYRLAENQKSCEPAVPFPCGRVSVSQTS
KLTRAETVFPDVDYVNSTEAETILDNITQSTQSFNDFTRVVGGEDAKPG
QFPWQVVLNGKVDAFCGGSIVNEKWIVTAAHCVETGVKITVVAGEHNIE
ETEHTEQKRNVIRIIPHHNYNAAINKYNHDIALLELDEPLVLNSYVTPI
CIADKEYTNIFLKFGSGYVSGWGRVFHKGRSALVLQYLRVPLVDRATCL
X₃₃₈STKFTIYNNMFCAGFHEGGRDSCQGDSGGPHVTEVEGTSFLTGII
SWGEECAMKGKYGIYTKVSRYVNWIKX₄₁₀KTKLT;

wherein X₈₅ is selected from D, F, G, H, I, M, N, R, S, W, and Y;

wherein X₈₆ is selected from A, D, E, G, H, I, L, M, N, P, Q, R, S, T, and V;

wherein X₈₇ is selected from F, I, K, M, R, T, V, and W;

wherein X₃₃₈ is selected from A, F, I, L, M, R, S, T, V, and W;

wherein X₄₁₀ is selected from E, N, and Q.

The introduction of at least one amino acid substitution is the result of a substitution at least one of the X positions. In some embodiments, the modified polypeptide additionally comprises between about 1-30, 1-20, or 1-10 conservative amino acid changes and maintains FIX activity. In some embodiments, the modified polypeptide is at least about 80, 85, 90, 95, or 99% identical to SEQ ID NO: 1 and maintains FIX activity.

### Production of Modified FIX Polypeptides

Amino acid sequence alteration may be accomplished by a variety of techniques, such as, for example, by modifying the corresponding nucleic acid sequence by site-specific mutagenesis. Techniques for site-specific mutagenesis are well known in the art and are described in, for example, Zoller, et al., (DNA 3:479-488, 1984) or Horton, et al., (Gene 77:61-68, 1989, pp. 61-68). Thus, using the nucleotide and amino acid sequences of FIX, one may introduce the alteration(s) of choice. Likewise, procedures for preparing a DNA construct using polymerase chain reaction using specific primers are well known to persons skilled in the art (see, e.g., PCR Protocols, 1990, Academic Press, San Diego, Calif., USA).

The nucleic acid construct encoding the FIX polypeptide may also be prepared synthetically by established standard methods, for example, the phosphoramidite method described by Beaucage, et al., (Gene Amplif. Anal. 3:1-26, 1983). According to the phosphoamidite method, oligonucleotides are synthesized, for example, in an automatic DNA synthesizer, purified, annealed, ligated, and cloned in suitable vectors. The DNA sequences encoding the FIX polypeptides may also be prepared by polymerase chain reaction using specific primers, for example, as described in U.S. Pat. No. 4,683,202; or Saiki, et al., (Science 239:487-491, 1988). Furthermore, the nucleic acid construct may be of mixed synthetic and genomic, mixed synthetic and cDNA, or mixed genomic and cDNA origin prepared by ligating fragments of synthetic, genomic, or cDNA origin (as appropriate), corresponding to various parts of the entire nucleic acid construct, in accordance with standard techniques.

The DNA sequences encoding the FIX polypeptides may be inserted into a recombinant vector using recombinant DNA procedures. The choice of vector will often depend on the host cell into which the vector is to be introduced. The vector may be an autonomously replicating vector or an integrating vector. An autonomously replicating vector exists as an extrachromosomal entity and its replication is independent of chromosomal replication, for example, a plasmid. An integrating vector is a vector that integrates into the host cell genome and replicates together with the chromosome(s) into which it has been integrated.

The vector may be an expression vector in which the DNA sequence encoding the modified FIX is operably linked to additional segments required for transcription, translation, or processing of the DNA, such as promoters, terminators, and polyadenylation sites. In general, the expression vector may be derived from plasmid or viral DNA, or may contain elements of both. The term “operably linked” indicates that the segments are arranged so that they function in concert for their intended purposes, for example, transcription initiates in a promoter and proceeds through the DNA sequence coding for the polypeptide.

Expression vectors for use in expressing FIX polypeptides may comprise a promoter capable of directing the transcription of a cloned gene or cDNA. The promoter may be any DNA sequence that shows transcriptional activity in the host cell of choice and may be derived from genes encoding proteins either homologous or heterologous to the host cell.

Examples of suitable promoters for directing the transcription of the DNA encoding the FIX polypeptides in mammalian cells are, for example, the SV40 promoter (Subramani, et al., Mol. Cell. Biol. 1:854-864, 1981), the MT-I (metallothionein gene) promoter (Palmiter, et al., Science 222:809-814, 1983), the CMV promoter (Boshart, et al., Cell 41:521-530, 1985), or the adenovirus 2 major late promoter (Kaufman et al., Mol. Cell. Biol, 2:1304-1319, 1982).

The DNA sequences encoding the FIX polypeptide may also, if necessary, be operably connected to a suitable terminator, such as the human growth hormone terminator (Palmiter, et al., Science 222:809-814, 1983) or TPIl (Alber et al., J. Mol. Appl. Gen. 1:419-434, 1982) or ADH3 (McKnight, et al., EMBO J. 4:2093-2099, 1985) terminators. The expression vectors may also contain a polyadenylation signal located downstream of the insertion site. Polyadenylation signals include the early or late polyadenylation signal from SV40, the polyadenylation signal from the adenovirus 5 EIb region, the human growth hormone gene terminator (DeNoto, et al., Nucl. Acids Res. 9:3719-3730, 1981), or the polyadenylation signal from the human FIX gene. The expression vectors may also include enhancer sequences, such as the SV40 enhancer.

To direct the FIX polypeptides of the present invention into the secretory pathway of the host cells, the native FIX secretory signal sequence may be used. Alternatively, a secretory signal sequence (also known as a leader sequence, prepro sequence, or pre sequence) may be provided in the recombinant vector. The secretory signal sequence may be joined to the DNA sequences encoding the FIX analogues in the correct reading frame. Secretory signal sequences are commonly positioned 5′ to the DNA sequence encoding the peptide. Exemplary signal sequences include, for example, the MPIF-1 signal sequence and the stanniocalcin signal sequence.

The procedures used to ligate the DNA sequences coding for the FIX polypeptides, the promoter, and optionally the terminator and/or secretory signal sequence and to insert them into suitable vectors containing the information necessary for replication, are well known to persons skilled in the art (see, e.g., Sambrook et al., Molecular Cloning: A Laboratory Manual, Cold Spring Harbor, N.Y., 1989).

Methods of transfecting mammalian cells and expressing DNA sequences introduced into the cells are described in, for example, Kaufman, et al., (J. Mol. Biol. 159:601-621, 1982); Southern, et al., (J. Mol. Appl. Genet. 1:327-341, 1982); Loyter, et al., (Proc. Natl. Acad. Sci. USA 79:422-426, 1982); Wigler, et al., (Cell 14:725-731, 1978); Corsaro, et al., (Somatic Cell Genetics 7:603-616, 1981), Graham, et al., (Virology 52:456-467, 1973); and Neumann, et al., (EMBO J. 1:841-845, 1982). Cloned DNA sequences may be introduced into cultured mammalian cells by, for example, lipofection, DEAE-dextran-mediated transfection, microinjection, protoplast fusion, calcium phosphate precipitation, retroviral delivery, electroporation, sonoporation, laser irradiation, magnetofection, natural transformation, and biolistic transformation (see, e.g., Mehier-Humbert, et al., Adv. Drug Deliv. Rev. 57:733-753, 2005). To identify and select cells that express the exogenous DNA, a gene that confers a selectable phenotype (a selectable marker) is generally introduced into cells along with the gene or cDNA of interest. Selectable markers include, for example, genes that confer resistance to drugs such as neomycin, puromycin, hygromycin, and methotrexate. The selectable marker may be an amplifiable selectable marker, which permits the amplification of the marker and the exogenous DNA when the sequences are linked Exemplary amplifiable selectable markers include dihydrofolate reductase (DHFR) and adenosine deaminase. It is within the purview of one skilled in the art to choose suitable selectable markers (see, e.g., U.S. Pat. No. 5,238,820).

After cells have been transfected with DNA, they are grown in an appropriate growth medium to express the gene of interest. As used herein the term “appropriate growth medium” means a medium containing nutrients and other components required for the growth of cells and the expression of the active FIX polypeptides.

Media generally include, for example, a carbon source, a nitrogen source, essential amino acids, essential sugars, vitamins, salts, phospholipids, protein, and growth factors, and in the case of vitamin K dependent proteins such as FIX, vitamin K may also be provided. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the FIX polypeptide.

Examples of mammalian cell lines for use in the present invention are the COS-1 (ATCC CRL 1650), baby hamster kidney (BHK), HKB11 cells (Cho, et al., J. Biomed. Sci, 9:631-638, 2002), and HEK-293 (ATCC CRL 1573; Graham, et al., J. Gen. Virol. 36:59-72, 1977) cell lines. In addition, a number of other cell lines may be used within the present invention, including rat Hep I (rat hepatoma; ATCC CRL 1600), rat Hep II (rat hepatoma; ATCC CRL 1548), TCMK-1 (ATCC CCL 139), Hep-G2 (ATCC HB 8065), NCTC 1469 (ATCC CCL 9.1), CHO-K¹ (ATCC CCL 61), and CHO-DUKX cells (Urlaub and Chasin, Proc. Natl. Acad. Sci. USA 77:4216-4220, 1980).

FIX polypeptides may be recovered from cell culture medium and may then be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation)), extraction (see, e.g., Protein Purification, Janson and Lars Ryden, editors, VCH Publishers, New York, 1989), or various combinations thereof. In an exemplary embodiment, the polypeptides may be purified by affinity chromatography on an anti-FIX antibody column. Additional purification may be achieved by conventional chemical purification means, such as high performance liquid chromatography. Other methods of purification are known in the art, and may be applied to the purification of the modified FIX polypeptides (see, e.g., Scopes, R., Protein Purification, Springer-Verlag, N.Y., 1982).

Generally, “purified” shall refer to a protein or peptide composition that has been subjected to fractionation to remove various other components, and which substantially retains its expressed biological activity. Where the term “substantially purified” is used, this designation shall refer to a composition in which the protein or peptide forms the major component of the composition, such as constituting about 50%, about 60%, about 70%, about 80%, about 90%, about 95%, about 99%, or more of the proteins in the composition.

Various methods for quantifying the degree of purification of the polypeptide are known to those of skill in the art. These include, for example, determining the specific activity of an active fraction, or assessing the amount of polypeptides within a fraction by SDS/PAGE analysis. An exemplary method for assessing the purity of a fraction is to calculate the specific activity of the fraction, compare the activity to the specific activity of the initial extract, and to thus calculate the degree of purity, herein assessed by a “-fold purification number.” The actual units used to represent the amount of activity will, of course, be dependent upon the particular assay technique.

In some embodiments, FIX polypeptides are recombinantly expressed in tissue culture cells and glycosylation is the result of the normal post-translational cell functioning of the host cell, such as a mammalian cell. In other instances, cells have been genetically engineered to express a combination of enzymes and desired polypeptides such that addition of a desired sugar moiety to an expressed polypeptide occurs within the cell. Alternatively, glycosylation may be achieved through chemical or enzymatic modification (see, e.g., Lee, et al., J. Biol. Chem. 264:13848-13855, 1989). A variety of methods have been proposed in the art to customize the glycosylation pattern of a polypeptide (see, e.g., WO 99/22764; WO 98/58964; WO 99/54342; US Publication No. 2008/0050772; and U.S. Pat. No. 5,047,335).

### Polymer Conjugation

The modified FIX polypeptides may further comprise one or more polymer conjugation sites that may be used for attaching a polymer moiety. In some embodiments, FIX polypeptides may be conjugated to a biocompatible polymer. The biocompatible polymer may be selected to provide the desired improvement in pharmacokinetics. For example, the identity, size, and structure of the polymer may be selected so as to improve the circulation half-life of the polypeptide having FIX activity or decrease the antigenicity of the polypeptide without an unacceptable decrease in activity.

The modified FIX polypeptide may include one or more sugar moieties that are naturally attached to the peptide during expression in mammalian cells. In some embodiments, the sugar moieties may serve as conjugation sites for attaching a polymer moiety. In some embodiments, the polymer moiety may be attached to the sugar moiety using various linkers or linkage chemistries. For example, the polymer moiety may be conjugated to the sugar moiety by a hydrazone linkage or an amino-oxy linkage.

Examples of polymers useful in the invention include, but are not limited to, poly(alkylene glycols) such as polyethylene glycol (PEG), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(alpha-hydroxy acid), poly(vinyl alcohol), polyphosphazene, polyoxazoline, poly(N-acryloylmorpholine), polysialic acid, hydroxyethyl starch (HES), polyethylene oxide, alkyl-polyethylene oxides, bispolyethylene oxides, co-polymers or block co-polymers of polyalkyene oxides, poly(ethylene glycol-co-propylene glycol), poly(N-2-(hydroxyproply)methyacrylamide), and dextran.

The polymer is not limited to a particular structure and may be linear (e.g., alkoxy PEG or bifunctional PEG), or non-linear such as branched, forked, multi-armed (e.g., PEGs attached to a polyol core), and dendritic. Moreover, the internal structure of the polymer may be organized in any number of different patterns and may be selected from the group consisting of homopolymer, alternating copolymer, random copolymer, block copolymer, alternating tripolymer, random tripolymer, and block tripolymer.

PEG and other water-soluble polymers (i.e., polymeric reagents) may be activated with a suitable activating group appropriate for coupling to a desired site on the FIX polypeptide. Thus, a polymeric reagent will possess a reactive group for reaction with the FIX polypeptide. Representative polymeric reagents and methods for conjugating these polymers to an active moiety are known in the art and further described in Zalipsky, et al., (“Use of Functionalized Poly(Ethylene Glycols) for Modification of Polypeptides” in Polyethylene Glycol Chemistry: Biotechnical and Biomedical Applications, J. M. Harris, Plenus Press, New York (1992)), and Zalipsky (Adv. Drug Rev. 16:157-182, 1995)

The weight-average molecular weight of the polymer may be from about 100 Daltons to about 150,000 Daltons. Exemplary ranges, however, include weight-average molecular weights in the range of greater than about 5,000 Daltons to about 100,000 Daltons, in the range of from about 6,000 Daltons to about 90,000 Daltons, in the range of from about 10,000 Daltons to about 85,000 Daltons, in the range of greater than about 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons.

Exemplary weight-average molecular weights for the biocompatible polymer include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. Branched versions of the biocompatible polymer (e.g., a branched 40,000 Dalton polymer comprised of two 20,000 Dalton polymers) having a total molecular weight of any of the foregoing can also be used.

In some embodiments, the polymer is PEG. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and may be represented by the formula: X—O(CH₂CH₂O)ₙ₋₁CH₂CH₂OH, where n is 20 to 2300 and X is H or a terminal modification, for example, a C₁₋₄ alkyl. PEG may contain further chemical groups which are necessary for binding reactions, which result from the chemical synthesis of the molecule, or which act as a spacer for optimal distance of parts of the molecule. In addition, such a PEG may consist of one or more PEG side-chains which are linked together. PEGs with more than one PEG chain are called multiarmed or branched PEGs. Branched PEGs may be prepared, for example, by the addition of polyethylene oxide to various polyols including glycerol, pentaerythriol, and sorbitol. For example, a four-armed branched PEG may be prepared from pentaerythriol and ethylene oxide. Examples of branched PEG are described in, for example, European Published Application No. 473084A and U.S. Pat. No. 5,932,462. One form of PEG includes two PEG side-chains (PEG2) linked via the primary amino groups of a lysine (Monfardini, et al., Bioconjugate Chem. 6:62-69, 1995).

In one embodiment, the polymer may be an end-capped polymer, that is, a polymer having at least one terminus capped with a relatively inert group, such as a lower C₁₋₆ alkoxy group, although a hydroxyl group may also be used. When the polymer is PEG, for example, a methoxy-PEG (commonly referred to as mPEG) which is a linear form of PEG wherein one terminus of the polymer has a methoxy (—OCH₃) group, while the other terminus is a hydroxyl or other functional group that may be optionally chemically modified may be used.

Multi-armed or branched PEG molecules, such as those described in U.S. Pat. No. 5,932,462, may also be used as the PEG polymer. In addition, the PEG may comprise a forked PEG (see, e.g., PCT Publication No. WO 1999/45964, discloses various forked PEG structures capable of use in one or more embodiments of the present invention). The chain of atoms linking the Z functional groups to the branching carbon atom serve as a tethering group and may comprise, for example, alkyl chains, ether chains, ester chains, amide chains, and combinations thereof.

The PEG polymer may also comprise a pendant PEG molecule having reactive groups, such as carboxyl, covalently attached along the length of the PEG rather than at the end of the PEG chain. The pendant reactive groups may be attached to the PEG directly or through a spacer moiety, such as an alkylene group.

To effect covalent attachment of the polymer molecule(s) to the polypeptide, the hydroxyl end groups of the polymer molecule must be provided in activated form, that is, with reactive functional groups (examples of which include primary amino groups, hydrazide (HZ), thiol, succinate (SUC), succinimidyl succinate (SS), succinimidyl succinamide (SSA), succinimidyl propionate (SPA), succinimidyl butanoate (SBA), succinimidyl carboxymethylate (SCM), benzotriazole carbonate (BTC), N-hydroxysuccinimide (NHS), aldehyde, nitrophenylcarbonate (NPC), and tresylate (TRES)). Suitably activated polymer molecules are commercially available, for example, NOF, Japan; Nektar Therapeutics, Inc., Huntsville, Ala.; PolyMASC Pharmaceuticals plc, UK; or SunBio Corporation, Anyang City, South Korea. Alternatively, the polymer molecules may be activated by conventional methods known in the art (see, e.g., WO 90/13540). Specific examples of activated linear or branched polymer molecules suitable for use in the present invention are commercially available, for example, NOF, Japan; Nektar Therapeutics, Inc., Huntsville, Ala. Specific examples of activated PEG polymers include the following linear PEGs: NHS-PEG, SPA-PEG, SSPA-PEG, SBA-PEG, SS-PEG, SSA-PEG, SC-PEG, SG-PEG, SCM-PEG, NOR-PEG, BTC-PEG, EPDX-PEG, NCO-PEG, NPC-PEG, CDI-PEG, ALD-PEG, TRES-PEG, VS-PEG, OPSS-PEG, IODO-PEG, and MAL-PEG, and branched PEGs, such as PEG2-NHS, PEG2-MAL, and those disclosed in, for example, U.S. Pat. No. 5,932,462 and U.S. Pat. No. 5,643,575, both of which are incorporated herein by reference.

In one embodiment, the polymer has a sulfhydryl reactive moiety that may react with a free cysteine on a FIX polypeptide to form a covalent linkage. Such sulfhydryl reactive moieties include thiol, triflate, tresylate, aziridine, oxirane, S-pyridyl, or maleimide moieties. Furthermore, the following publications, incorporated herein by reference, disclose useful polymer molecules and/or PEGylation chemistries: U.S. Pat. Nos. 6,113,906; 7,199,223; 5,824,778; 5,476,653; 4,902,502; 5,281,698; 5,122,614; 5,219,564; 5,736,625; 5,473,034; 5,516,673; 5,629,384; 5,382,657; WO 97/32607; WO 92/16555; WO 94/04193; WO 94/14758; WO 94/17039; WO 94/18247; WO 94/28024; WO 95/00162; WO 95/11924; WO95/13090; WO 95/33490; WO 96/00080; WO 97/18832; WO 98/41562; WO 98/48837; WO 99/32134; WO 99/32139; WO 99/32140; WO 96/40791; WO 98/32466; WO 95/06058; WO 97/03106; WO 96/21469; WO 95/13312; WO 98/05363; WO 96/41813; WO 96/07670; EP809996; EP921131; EP605963; EP510356; EP400472; EP183503; EP154316; EP229108; EP402378; and EP439508.

For PEGylation of cysteine residues, the polypeptide may be treated with a reducing agent, such as dithiothreitol (DDT) prior to PEGylation. The reducing agent may be subsequently removed by any conventional method, such as by desalting. Conjugation of PEG to a cysteine residue typically takes place in a suitable buffer at pH 6-9 at temperatures varying from 4° C. to 25° C. for periods up to about 16 hours. Examples of activated PEG polymers for coupling to cysteine residues include, for example, the following linear and branched PEGs: vinylsulfone-PEG (PEG-VS), such as vinylsulfone-mPEG (mPEG-VS); orthopyridyl-disulfide-PEG (PEG-OPSS), such as orthopyridyl-disulfide-mPEG (MPEG-OPSS); and maleimide-PEG (PEG-MAL), such as maleimide-mPEG (mPEG-MAL) and branched maleimide-mPEG2 (mPEG2-MAL).

In one embodiment, FIX polypeptides having one or more introduced polymer conjugation sites may be expressed in cells grown in cell culture medium containing cysteines that “cap” the cysteine residues of the polypeptide by forming disulfide bonds. To add a polymer conjugate to the FIX polypeptides, the cysteine cap may be removed by mild reduction that releases the cap, and then a cysteine-specific polymer reagent is added.

The application also provides a method for the preparation of a polymer conjugated FIX polypeptide comprising introducing a polymer conjugation site, that is, a cysteine residue into a nucleotide sequence that encodes a FIX polypeptide; expressing the mutated nucleotide sequence to produce a polypeptide comprising an introduced polymer conjugation site; purifying the polypeptide; reacting the polypeptide with a biocompatible polymer that has been activated to react with polypeptides at reduced cysteine residues such that a conjugate is formed; and purifying the conjugate. In another embodiment, the application provides a method for site-directed PEGylation of a FIX polypeptide mutein comprising: (a) expressing a FIX polypeptide comprising an introduced polymer conjugation site, that is, a cysteine residue introduced on the exposed surface of the FIX polypeptide, wherein the cysteine is capped; (b) contacting the FIX polypeptide with a reductant under conditions to mildly reduce the introduced cysteine and release the cap; (c) removing the cap and the reductant from the FIX polypeptide; and (d) at least about 5, 15, or 30 minutes after the removal of the reductant, treating the FIX polypeptide with PEG comprising a sulfhydryl coupling moiety under conditions such that PEGylated FIX polypeptide is produced. The sulfhydryl coupling moiety of the PEG is selected from the group consisting of thiol, triflate, tresylate, aziridine, oxirane, S-pyridyl, and maleimide moieties.

An exemplary method of producing a PEGylated FIX polypeptide is described below. About 1 μM of a purified FIX polypeptide comprising an introduced non-native cysteine residue is mildly reduced with reductants such as 0.7 mM Tris(2-carboxyethyl)phosphine (TCEP) or 0.07 mM dithiothreitol (DTT) for 30 minutes at 4° C. to release the “cap.” The reductant is removed along with the “cap” by a size-exclusion chromatography (SEC) method such as running the sample through a spin column to allow disulfides to reform while leaving the introduced cysteine free and reduced. At least 30 minutes after the removal of the reductant, the FIX polypeptide is treated with at least 10-fold molar excess of PEG-maleimide with sizes ranging from 5 to 85 kD for at least 1 hour at 4° C.

Polymer conjugation of FIX may be assessed by any of the methods known to one of skill in the art. For example, polymer conjugated FIX may be analyzed by electrophoresis on a reducing 6% Tris-Glycine SDS polyacrylamide gel. Following electrophoresis, the gel may be stained with Coomassie Blue to identify all the proteins or subjected to a standard western blot protocol, in order to identify shifts in band molecular weight as compared to unconjugated FIX polypeptides. Barium-iodine staining which is specific for PEG, may be used to confirm that bands with a shift in molecular weight comprise a PEGylated protein. FIX polypeptides, before and after polymer conjugation, may also be analyzed by matrix-assisted laser desorption/ionization (MALDI) mass spectrometry, in order to determine the extent and efficiency of polymer conjugation.

In some embodiments, polymer conjugation may occur on one or more of the sugar moieties attached by glycosylation. Methods of such polymer conjugation are known in the art and have been described for example in WO94/05332, US2009/0081188 and U.S. Pat. No. 5,621,039, both of which are incorparated by reference. Where the polymer is PEG, it is also commonly referred to as glycoPEGylation.

In some embodiments, polymer conjugation by chemical attachment as provided in U.S. Pat. No. 5,621,039 can be improved by the addition of a catalyst. In some embodiments, the catalyst is a chemical catalyst. For example, the chemical catalyst may be aniline, which can be used to increase the efficiency of a reaction between a free aldehyde on sugars and an amino group. In other embodiments, other suitable chemical catalysts may be aniline derivatives such as o-Cl-, p-Cl-, o-CH3O-, p-CH3O-, and p-CH3-analine.

In some embodiments, polymer conjugation may occur at naturally occurring glycosylation sites in FIX. Wild type Factor IX has two N-linked glycosylation sites that contain about 80% of the total sialic acid content of Factor IX. These two N-linked sites (N157 and N167) are both located within the activation peptide that is cleaved at two sites (R145-Ala146) and (R180-V181) to generate the catalytically active FIXa molecule during the propagation of the coagulation cascade.

In addition to polymer conjugation at naturally occurring glycosylation sites in FIX it may be desirable to conjugate polymers, in at alternative sites located in different domains of the FIX protein. This can be achieved by first ablating the naturally occurring N-linked glycosylation sites at positions N157 and N167 by for example changing N157 to A157 and N167 to A167 and secondly by introducing a novel and functional N-linked glycosylation site elsewhere in the molecule, for example in the catalytic domain or one of the two EGF domains. Such novel and functional N-linked glycosylation sites have been previously disclosed in PCT US2009/040813.

### Pharmaceutical Compositions

Based on well known assays used to determine the efficacy for treatment of conditions identified above in mammals, and by comparison of these results with the results of known medicaments that are used to treat these conditions, the effective dosage of the polypeptides of this invention may readily be determined for treatment of each desired indication. The amount of the active ingredient to be administered in the treatment of one of these conditions can vary widely according to such considerations as the particular polypeptide and dosage unit employed, the mode of administration, the period of treatment, the age and sex of the patient treated, and the nature and extent of the condition treated.

The application provides, in part, compositions comprising FIX polypeptides with one or more amino acid substitutions as described herein. The compositions may be suitable for in vivo administration and are pyrogen free. The compositions may also comprise a pharmaceutically acceptable carrier. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Supplementary active ingredients also may be incorporated into the compositions.

The compositions of the present invention include classic pharmaceutical preparations. Administration of these compositions according to the present invention may be via any common route. The pharmaceutical compositions may be introduced into the subject by any conventional method, for example, by intravenous, intradermal, intramuscular, subcutaneous, or transdermal delivery. The treatment may consist of a single dose or a plurality of doses over a period of time.

The active compounds may be prepared for administration as solutions of free base or pharmacologically acceptable salts in water. Dispersions also may be prepared in liquid polyethylene glycols. Under ordinary conditions of storage and use, these preparations contain a preservative to prevent the growth of microorganisms.

The pharmaceutical forms, suitable for injectable use, include sterile aqueous solutions or dispersions and sterile powders for the extemporaneous preparation of sterile injectable solutions or dispersions. The form should be sterile and should be fluid to the extent that easy syringability exists. It should be stable under the conditions of manufacture and storage and should be preserved against the contaminating action of microorganisms, such as bacteria and fungi. The carrier may be a solvent or dispersion medium containing, for example, water, ethanol, polyol (e.g., glycerol, propylene glycol, and liquid polyethylene glycol, and the like) sucrose, L-histidine, polysorbate 80, or suitable mixtures thereof. The prevention of the action of microorganisms may be brought about by various antibacterial an antifungal agents, for example, parabens, chlorobutanol, phenol, sorbic acid, thimerosal, and the like. The injectable compositions may include isotonic agents, for example, sugars or sodium chloride. Prolonged absorption of the injectable compositions may be brought about by the use in the compositions of agents delaying absorption.

Sterile injectable solutions may be prepared by incorporating the active compounds (e.g., FIX polypeptides) in the required amount in the appropriate solvent with various of the other ingredients enumerated above, as required, followed by filtered sterilization.

Generally, dispersions may be prepared by incorporating the various sterilized active ingredients into a sterile vehicle that contains the basic dispersion medium and the required other ingredients from those enumerated above. In the case of sterile powders for the preparation of sterile injectable solutions, methods of preparation include, for example, vacuum-drying and freeze-drying techniques that yield a powder of the active ingredient plus any additional desired ingredient from a previously sterile-filtered solution thereof.

The composition may also include an antimicrobial agent for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant may be present in the composition as well. Antioxidants may be used to prevent oxidation, thereby preventing the deterioration of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates such as Tween®-20 (polyoxyethylenesorbitan monolaurate) and Tween®-80 (polyoxyethylenesorbitan monooleate) and pluronics such as F68 and F88 (both of which are available from BASF, Mount Olive, N.J.); sorbitan esters; lipids such as phospholipids such as lecithin and other phosphatidylcholines, phosphatidylethanolamines, fatty acids and fatty esters; steroids such as cholesterol; and chelating agents such as EDTA, zinc and other such suitable cations.

Acids or bases may be present as an excipient in the composition. Non-limiting examples of acids that may be used include hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of any individual excipient in the composition may vary depending on the activity of the excipient and particular needs of the composition. Typically, the optimal amount of any individual excipient may be determined through routine experimentation, that is, by preparing compositions containing varying amounts of the excipient (ranging from low to high), examining the stability and other parameters, and then determining the range at which optimal performance is attained with no significant adverse effects. Generally, the excipient may be present in the composition in an amount of about 1% to about 99% by weight, from about 5% to about 98% by weight, from about 15 to about 95% by weight of the excipient, with concentrations less than 30% by weight. These foregoing pharmaceutical excipients along with other excipients are described in “Remington: The Science & Practice of Pharmacy,” 19 ed., Williams & Williams, (1995); the “Physician's Desk Reference,” 52 ed., Medical Economics, Montvale, N.J. (1998); and Kibbe, A. H., Handbook of Pharmaceutical Excipients, 3 Edition, American Pharmaceutical Association, Washington, D.C., 2000.

Upon formulation, solutions may be administered in a manner compatible with the dosage formulation and in such amount as is therapeutically effective. “Therapeutically effective amount” is used herein to refer to the amount of a polypeptide that is needed to provide a desired level of the polypeptide in the bloodstream or in the target tissue. The precise amount will depend upon numerous factors, for example, the particular FIX polypeptide, the components and physical characteristics of the therapeutic composition, intended patient population, mode of delivery, individual patient considerations, and the like, and can readily be determined by one skilled in the art, based upon the information provided herein.

The formulations may be easily administered in a variety of dosage forms, such as injectable solutions, and the like. For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous and intraperitoneal administration.

Dosages of FIX are normally expressed in units. One unit of FIX per kg of body weight may raise plasma levels by 0.01 U/ml, that is, 1%. Otherwise healthy patients have one unit of FIX per ml of plasma, that is, 100%. Mild cases of hemophilia B are defined by FIX plasma concentrations between 6-60%, moderate cases between 1-5%, and severe cases, which account for about half of the hemophilia B cases, have less than 1% FIX. Prophylactic treatment or treatment of minor hemorrhaging usually requires raising FIX levels to between 15-30%. Treatment of moderate hemorrhaging usually requires raising levels to between 30-50%, while treatment of major trauma may require raising levels from 50 to 100%. The total number of units needed to raise a patient's blood level can be determined as follows: 1.0 unit/kg×body weight (kg)×desired percentage increase (% of normal). Parenteral administration may be carried out with an initial bolus followed by continuous infusion to maintain therapeutic circulating levels of drug product. In some embodiments, between 15 to 150 units/kg of FIX polypeptide may be administered. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation may be determined by one of skill in the art depending on the route of administration and the desired dosage (see, e.g., Remington's Pharmaceutical Sciences, Mack Publishing Co., Easton, Pa., 20^{th} edition, 2000, incorporated herein by reference). Such formulations may influence the physical state, stability, rate of in vivo release, and rate of in vivo clearance of the administered agents. Depending on the route of administration, a suitable dose may be calculated according to body weight, body surface area, or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials. Exemplary dosing schedules include, without limitation, administration five times a day, four times a day, three times a day, twice daily, once daily, three times weekly, twice weekly, once weekly, twice monthly, once monthly, and any combination thereof.

Appropriate dosages may be ascertained through the use of established assays for determining blood clotting levels in conjunction with relevant dose response data. The final dosage regimen may be determined by the attending physician, considering factors that modify the action of drugs, for example, the drug's specific activity, severity of the damage, and the responsiveness of the patient, the age, condition, body weight, sex and diet of the patient, the severity of any infection, time of administration, and other clinical factors.

### Exemplary Uses

The compositions described herein may be used to treat any bleeding disorder associated with functional defects of FIX or deficiencies of FIX such as a shortened in vivo half-life of FIX, altered binding properties of FIX, genetic defects of FIX, and a reduced plasma concentration of FIX. Genetic defects of FIX comprise, for example, deletions, additions, and/or substitution of bases in the nucleotide sequence encoding FIX. In one embodiment, the bleeding disorder may be hemophilia B. Symptoms of such bleeding disorders include, for example, severe epistaxis, oral mucosal bleeding, hemarthrosis, hematoma, persistent hematuria, gastrointestinal bleeding, retroperitoneal bleeding, tongue/retropharyngeal bleeding, intracranial bleeding, and trauma-associated bleeding.

The compositions of the present invention may be used for prophylactic applications. In some embodiments, modified FIX polypeptides may be administered to a subject susceptible to or otherwise at risk of a disease state or injury to enhance the subject's own coagulative capability. Such an amount may be defined to be a “prophylactically effective dose.” Administration of the modified FIX polypeptides for prophylaxis includes situations where a patient suffering from hemophilia B is about to undergo surgery and the polypeptide is administered between one to four hours prior to surgery. In addition, the polypeptides are suited for use as a prophylactic against uncontrolled bleeding, optionally in patients not suffering from hemophilia. Thus, for example, the polypeptide may be administered to a patient at risk for uncontrolled bleeding prior to surgery.

The polypeptides, materials, compositions, and methods described herein are intended to be representative examples of the invention, and it will be understood that the scope of the invention is not limited by the scope of the examples. Those skilled in the art will recognize that the invention may be practiced with variations on the disclosed polypeptides, materials, compositions and methods, and such variations are regarded as within the ambit of the invention.

The following examples are presented to illustrate the invention described herein, but should not be construed as limiting the scope of the invention in any way.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Cloning of human Factor IX cDNA

A pair of PCR primers complementary to sequences at the 5′ and 3′ ends of the coding region of the human FIX cDNA were designed from the published cDNA sequence (NM_{—}000133). The 5′ primer (FIXF1; ATCATAAGCTTGCCACCATGCAGCGCGTGAACATG (SEQ ID NO: 3), start codon of FIX is in bold text) contained the first 18 nucleotides of the FIX coding region including the ATG start codon preceded by a consensus Kozak sequence (underlined) and a HindIII restriction site. The 3′ primer (FIXR3, ATCATAAGCTTGATTAGTTAGTGAGAGGCC CTG) (SEQ ID NO: 4) contained 22 nucleotides of FIX sequence that lies 45 nucleotides 3′ of the end of the FIX coding region preceded by a HindIII site. Amplification of first strand cDNA from normal human liver (Stratagene, San Diego, Calif.) using these primers and high fidelity proofreading polymerase (Invitrogen, Carlsbad, Calif.) resulted in a single band of the expected size for human FIX cDNA (1464 bp). After digestion with HindIII, the PCR product was gel purified and then cloned into the HindIII site of the plasmid pEAKflcmv. Clones in which the FIX cDNA was inserted in the forward orientation relative to the CMV promoter in the vector were identified by restriction digest. Double stranded DNA sequencing was performed for the insert of several clones and alignment of the derived sequence to the FIX sequence demonstrated that the cDNA encodes human FIX with threonine at amino acid 148 of the mature protein. This plasmid was designated as pEAKflcmv-FIX.

### Example 2

### Generation of Modified Factor IX Polypeptides

To change various amino acids within the human FIX sequence, a pair of primers were designed using the Quickchange™ primer design program (Stratagene, San Diego, Calif.). These primers were used to generate mutations in the pEAKflcmv-FIX plasmid employing the Quickchange™ II XL site directed mutagenesis kit (Stratagene, San Diego, Calif.) according to the manufacturer's instructions. Clones containing the desired mutation were identified by DNA sequencing of the entire FIX coding region. The sequence of the sense strand oligonucleotide used to create the mutations is shown in Table 1.

TABLE 1

| Substitution | Sense Strand Oligonucleotide Sequence |
|---|---|
| V86A | f: AGGAAAGAACTGTGAATTAGATGCCACATGTAACATTAAGAA |
| | TGGCA (SEQ ID NO: 5) |
| | r: TGCCATTCTTAATGTTACATGTGGCATCTAATTCACAGTTCTTTCCT |
| | (SEQ ID NO: 6) |
| V86P | f: GGAAAGAACTGTGAATTAGATCCCACATGTAACATTAAGAAT |
| | GGCAG (SEQ ID NO: 7) |
| | r: CTGCCATTCTTAATGTTACATGTGGGATCTAATTCACAGTTCTTTCC |
| | (SEQ ID NO: 8) |
| V86E | f: GGAAAGAACTGTGAATTAGATGAGACCTGTAACATTAAGAATG |
| | GCAG (SEQ ID NO: 9) |
| | r: CTGCCATTCTTAATGTTACAGGTCTCATCTAATTCACAGTTCTTTCC |
| | (SEQ ID NO: 10) |
| V86S | f: GGAAAGAACTGTGAATTAGATAGCACCTGTAACATTAAGAATG |
| | GCAG (SEQ ID NO: 11) |
| | r: CTGCCATTCTTAATGTTACAGGTGCTATCTAATTCACAGTTCTTTCC |
| | (SEQ ID NO: 12) |
| V86I | f: GGAAAGAACTGTGAATTAGATATCACCTGTAACATTAAGAATG |
| | GCAG (SEQ ID NO: 13) |
| | r: CTGCCATTCTTAATGTTACAGGTGATATCTAATTCACAGTTCTTTCC |
| | (SEQ ID NO: 14) |
| V86R | f:GGAAAGAACTGTGAATTAGATAGAACCTGTAACATTAAGAATG |
| | GCAG (SEQ ID NO: 15) |
| | r: CTGCCATTCTTAATGTTACAGGTTCTATCTAATTCACAGTTCTTTCC |
| | (SEQ ID NO: 16) |
| V86Q | f:GGAAAGAACTGTGAATTAGATCAGACATGTAACATTAAGAATG |
| | GCAG (SEQ ID NO: 17) |
| | r: CTGCCATTCTTAATGTTACATGTCTGATCTAATTCACAGTTCTTTCC |
| | (SEQ ID NO: 18) |
| V86T | f: GGAAAGAACTGTGAATTAGATACCACCTGTAACATTAAGAATG |
| | GCAG (SEQ ID NO: 19) |
| | r: CTGCCATTCTTAATGTTACAGGTGGTATCTAATTCACAGTTCTTTCC |
| | (SEQ ID NO: 20) |
| V86D | f: GGAAAGAACTGTGAATTAGATGACACCTGTAACATTAAGAATG |
| | GCAG (SEQ ID NO: 21) |
| | r: CTGCCATTCTTAATGTTACAGGTGTCATCTAATTCACAGTTCTTTCC |
| | (SEQ ID NO: 22) |
| V86H | f: GGAAAGAACTGTGAATTAGATCACACCTGTAACATTAAGAATG |
| | GCAG (SEQ ID NO: 23) |
| | r: CTGCCATTCTTAATGTTACAGGTGTGATCTAATTCACAGTTCTTTCC |
| | (SEQ ID NO: 24) |
| V86N | f: GGAAAGAACTGTGAATTAGATAACACCTGTAACATTAAGAATG |
| | GCAG (SEQ ID NO: 25) |
| | r: CTGCCATTCTTAATGTTACAGGTGTTATCTAATTCACAGTTCTTTCC |
| | (SEQ ID NO: 26) |
| V86L | f:GGAAAGAACTGTGAATTAGATCTGACATGTAACATTAAGAATG |
| | GCAG (SEQ ID NO: 27) |
| | r: CTGCCATTCTTAATGTTACATGTCAGATCTAATTCACAGTTCTTTCC |
| | (SEQ ID NO: 28) |
| V86M | f: GGAAAGAACTGTGAATTAGATATGACCTGTAACATTAAGAATG |
| | GCAG (SEQ ID NO: 29) |
| | r: CTGCCATTCTTAATGTTACAGGTCATATCTAATTCACAGTTCTTTCC |
| | (SEQ ID NO: 30) |
| V86Y | f: GGAAAGAACTGTGAATTAGATTACACCTGTAACATTAAGAATG |
| | GCAG (SEQ ID NO: 31) |
| | r: CTGCCATTCTTAATGTTACAGGTGTAATCTAATTCACAGTTCTTTCC |
| | (SEQ ID NO: 32) |
| V86K | f: GGAAAGAACTGTGAATTAGATAAGACATGTAACATTAAGAATG |
| | GCAG (SEQ ID NO: 33) |
| | r: CTGCCATTCTTAATGTTACATGTCTTATCTAATTCACAGTTCTTTCC |
| | (SEQ ID NO: 34) |
| V86F | f: GGAAAGAACTGTGAATTAGATTTCACCTGTAACATTAAGAATG |
| | GCAG (SEQ ID NO: 35) |
| | r: CTGCCATTCTTAATGTTACAGGTGAAATCTAATTCACAGTTCTTTCC |
| | (SEQ ID NO: 36) |
| V86C | f: GGAAAGAACTGTGAATTAGATTGCACCTGTAACATTAAGAATG |
| | GCAG (SEQ ID NO: 37) |
| | r: CTGCCATTCTTAATGTTACAGGTGCAATCTAATTCACAGTTCTTTCC |
| | (SEQ ID NO: 38) |
| V86W | f: GGAAAGAACTGTGAATTAGATTGGACCTGTAACATTAAGAATG |
| | GCAG (SEQ ID NO: 39) |
| | r: CTGCCATTCTTAATGTTACAGGTCCAATCTAATTCACAGTTCTTTCC |
| | (SEQ ID NO: 40) |
| V86G | f: GAAGGAAAGAACTGTGAATTAGATGGCACCTGTAACATTAAGAAT |
| | GGCAGATGCG (SEQ ID NO: 41) |
| | r: CGCATCTGCCATTCTTAATGTTACAGGTGCCATCTAATTCACAGTTC |
| | TTTCCTTC (SEQ ID NO: 42) |
| E410N | f: TCCCGGTATGTCAACTGGATTAAGAACAAAACAAAGCTCACTTAA |
| | TGAAAG (SEQ ID NO: 43) |
| | r: CTTTCATTAAGTGAGCTTTGTTTTGTTCTTAATCCAGTTGACATACC |
| | GGGA (SEQ ID NO: 44) |
| E410Q | f: TCCCGGTATGTCAACTGGATTAAGCAGAAAACAAAGCTCACTTAA |
| | TGAAAG (SEQ ID NO: 45) |
| | r: CTTTCATTAAGTGAGCTTTGTTTTCTGCTTAATCCAGTTGACATACC |
| | GGGA (SEQ ID NO: 46) |
| N157A | CTGTTTTTCCTGATGTGGACTACGTAGCCTCTACTGAAGCTGAAACCATTCT |
| | (SEQ ID NO: 47) |
| N167A | GAAGCTGAAACCATTCTAGATGCCATCACTCAAAGCACCCAATC (SEQ ID |
| | NO: 48) |
| R338A | CTTGTTGACCGAGCCACATGCCTTGCATCTACAAAGTTCACCATC (SEQ ID |
| | NO: 49) |
| R338L | CTTGTTGACCGAGCCACATGCCTTCTGTCTACAAAGTTCACCATC (SEQ ID |
| | NO: 50) |
| R338V | GACCGAGCCACATGCCTTGTGTCTACAAAGTTCACCATC (SEQ ID NO: 51) |
| R338I | GTTGACCGAGCCACATGCCTTATCTCTACAAAGTTCACCATCTATAAC (SEQ |
| | ID NO: 52) |
| R338F | GTTGACCGAGCCACATGCCTTTTCTCTACAAAGTTCACCATCTATAAC (SEQ |
| | ID NO: 53) |
| R338W | CTTGTTGACCGAGCCACATGCCTTTGGTCTACAAAGTTCACCATC (SEQ ID |
| | NO: 54) |
| R338M | CACTTGTTGACCGAGCCACATGCCTTATGTCTACAAAGTTCACCATC (SEQ ID |
| | NO: 55) |
| R338S | CTTGTTGACCGAGCCACATGCCTTAGCTCTACAAAGTTCACCATC (SEQ ID |
| | NO: 56) |
| R338T | GTTGACCGAGCCACATGCCTTACCTCTACAAAGTTCACCATC (SEQ ID NO: 57) |

### Example 3

### Expression of Factor IX Polypeptides in HKB11 Cells

In order to determine if the FIX genes with altered protein sequences could be expressed and secreted from mammalian cells and to determine the effect of these substitutions upon FIX coagulation activity, expression plasmids encoding these FIX variants were transfected into HKB11 cells. HKB11 is a human cell line generated by the fusion of HEK293 cells and a B cell lymphoma.

HKB11 cells were grown in suspension culture on an orbital shaker (100-125 rpm) in a CO₂ (5%) incubator at 37° C. in serum-free media supplemented with 10 ng/mL soluble vitamin K₃ (Sigma-Aldrich, St. Louis, Mo.) and maintained at a density between 0.25 and 1.5×10⁶ cells/mL.

Cells for transfection were collected by centrifugation at 1000 rpm for 5 minutes then resuspended in FreeStyle™ 293 Expression Medium (Invitrogen, Carlsbad, Calif.) at 1.1×10⁶ cells/mL. The cells were seeded in 6 well plates (4.6 mL/well) and incubated on an orbital rotator (125 rpm) in a 37° C. CO₂ incubator. For each well, 5 μg plasmid DNA was mixed with 0.2 mL Opti-MEM® I medium (Invitrogen). For each well, 7 μL 293Fectin™ reagent (Invitrogen) was mixed gently with 0.2 mL Opti-MEM® I medium and incubated at room temperature for 5 minutes. The diluted 293Fectin™ was added to the diluted DNA solution, mixed gently, incubated at room temperature for 20-30 minutes and then added to each well that had been seeded with 5×10⁶ (4.6 mL) HKB 11 cells. The cells were then incubated on an orbital rotator (125 rpm) in a CO₂ incubator at 37° C. for 3 days after which the cells were pelleted by centrifugation at 1000 rpm for 5 minutes, and the supernatant was collected and stored at 4° C.

### Example 4

### Expression of Factor IX Polypeptides in BHK21 Cells

In order to determine if the FIX genes with altered protein sequences could be expressed and secreted from mammalian cells and to determine the effect of these substitutions upon FIX coagulation activity, expression plasmids encoding these FIX variants were transfected into BHK21 cells.

BHK21 cells are grown in suspension culture on an orbital shaker (100-125 rpm) in a CO₂ (5%) incubator at 37° C. in a proprietary serum free media supplemented with 10 ng/ml soluble vitamin K3 (Menadione, Sigma) and maintained at a density between 0.25 and 1.5×10⁶ cells/ml.

Cells for transfection are collected by centrifugation at 1000 rpms for 5 minutes then resuspended at 1×10⁶ cells/ml.

The cells are seeded in 6 well plates (4.6 ml/well) and incubated on an orbital rotator (125 rpm) in a 37° C. CO₂ incubator. For each well, 5 μg of plasmid DNA is mixed with 0.2 ml Opti-MEM I medium (Invitrogen). For each well, 7 μl of 293Fectin reagent (Invitrogen) is mixed gently with 0.2 ml of Opti-MEM I medium and incubated at room temperature for 5 min. The diluted 293Fectin is added to the diluted DNA solution, mixed gently, incubated at room temperature for 20-30 minutes then added to each well that has been seeded with 5×10⁶ (4.6 ml) BHK21 cells. The cells are then incubated on an orbital rotator (125 rpm) in a CO₂ incubator at 37° C. for 3 days after which the cells are pelleted by centrifugation at 1000 rpm for 5 minutes and the supernatant is collected and stored at 4° C.

### Example 5

### Western Blot for Factor IX

Cell culture supernatant (50 μL) was mixed with 20 μL 4×SDS-PAGE loading dye, heated at 95° C. for 5 minutes, loaded on NuPAGE® 4-12% SDS PAGE gels and then transferred to nitrocellulose membranes. After blocking with 5% milk powder for 30 minutes, the membranes were incubated with a HRP-labeled goat polyclonal antibody against human FIX (US Biological, Swampscott, Mass., Catalog No. F0017-07B) for 60 minutes at room temperature. After washing with phosphate-buffered saline with 0.1% Tween®-20 buffer, the signal from HRP was detected using SuperSignal® Pico (Pierce, Rockford, Ill.) and exposure to x-ray film.

### Example 6

### Factor IX ELISA

FIX antigen levels in cell culture supernatants were determined using a FIX ELISA kit (Hyphen Biomed/Aniara, Mason, Ohio). Cell culture supernatant was diluted in sample diluent buffer (supplied in the kit) to achieve a signal within the range of the standard curve. FIX protein purified from human plasma (Hyphen Biomed/Aniara, Catalog No. RK032A, specific activity 196 U/mg) diluted in sample diluent was used as to create a standard curve from 100 ng/mL to 0.2 ng/mL. Diluted samples and the standards were added to the ELISA plate that is pre-coated with a polyclonal anti-FIX capture antibody. After adding the polyclonal detection antibody, the plate was incubated at room temperature for 1 hour, washed extensively, then developed using TMB substrate (3,3′,5,5′-tetramethylbenzidine) as described by the kit manufacturer and the signal is measured at 450 nM using a SpectraMax® plate reader (Molecular Devices, Sunnyvale, Calif.). The standard curve was fitted to a 2-component plot and the values of the unknowns extrapolated from the curve.

FIX expression levels were also quantitated using commercially available FIX ELISA reagents (Haemochrom Diagnostica GmbH, Essen, Germany) according to the manufacturer's instructions. Wheat germ agglutinin (Sigma-Aldrich, St. Louis, Mo.) was coated on 384 well MaxiSorp™ plates (Nunc™, Rochester, N.Y.). The wells were blocked, washed, and then supernatant was added. After further washing, detection was carried out using HRP-coupled polyclonal anti-FIX antibody (Haemochrom Diagnostica GmbH, Essen, Germany).

### Example 7

### Factor IX Coagulation Assay

FIX coagulation activity was determined using an aPTT assay in FIX deficient human plasma run on a Electra™ 1800C automatic coagulation analyzer (Beckman Coulter, Fullerton, Calif.). Briefly, three dilutions of supernatant samples in coagulation diluent were created by the instrument, and 100 μL was then mixed with 100 μL FIX deficient plasma (Aniara, Mason, Ohio) and 100 μL automated aPTT reagent (rabbit brain phospholipid and micronized silica (bioMérieux, Inc., Durham, N.C.). After the addition of 100 μL 25 mM CaCl₂ solution, the time to clot formation was recorded. A standard curve was generated for each run using serial dilutions of the same purified human FIX (Hyphen Biomed/Aniara) used as the standard in the ELISA assay. The standard curve was routinely a straight line with a correlation coefficient of 0.95 or better and was used to determine the FIX activity of the unknown samples. The activity for FIX polypeptides comprising an amino acid substitution at position 86 is shown in Table 2. The activity for FIX polypeptides comprising one or more amino acid substitutions is shown in Tables 3 and 4.

TABLE 2

| | Protein | | Specific |
|---|---|---|---|
| | expression | Activity | Activity |
| Factor IX | (% of wild | (% of wild | (% of wild |
| substitution | type) | type) | type) |
| Wild type | 100 | 100 | 100 |
| Factor IX | | | |
| V86A | 31 | 142 | 458 |
| V86P | 58 | 231 | 399 |
| V86E | 103 | 241 | 233 |
| V86S | 98 | 164 | 167 |
| V86I | 39 | 59 | 153 |
| V86Q | 88 | 109 | 123 |
| V86G | 102 | 122 | 115 |
| V86R | 78 | 82 | 105 |
| V86T | 81 | 60 | 74 |
| V86D | 68 | 45 | 67 |
| V86H | 71 | 41 | 57 |
| V86N | 89 | 42 | 47 |
| V86L | 90 | 26 | 28 |
| V86M | 72 | 19 | 26 |
| V86K | 68 | 12 | 17 |

TABLE 3

| | Protein | | Specific |
|---|---|---|---|
| | expression | Activity | Activity |
| Factor IX | (% of wild | (% of wild | (% of wild |
| substitution | type) | type) | type) |
| Wild type FIX | 100 | 100 | 100 |
| R338A | 95 | 395 | 450 |
| V86A | 120 | 205 | 180 |
| R338A/V86A | 55 | 550 | 1200 |
| R338A/V86P | 72 | 727 | 999 |
| R338A/V86E | 110 | 554 | 492 |
| R338A/V86S | 140 | 544 | 360 |
| R338A/V86A/ | 79 | 2150 | 2700 |
| E410N | | | |
| R338A/V86A/ | 66 | 2350 | 3550 |
| E410Q | | | |

TABLE 4

| Amino acid | | Fold activity |
|---|---|---|
| substitution | | over R338A/ |
| 85 | 87 | V86A |
| W | R | 3.15 |
| F | I | 2.30 |
| W | W | 2.30 |
| R | R | 2.21 |
| I | R | 2.06 |
| Y | F | 1.98 |
| I | M | 1.97 |
| F | R | 1.94 |
| F | V | 1.88 |
| R | K | 1.79 |
| H | I | 1.72 |
| I | I | 1.72 |
| Y | K | 1.71 |
| S | R | 1.71 |
| Y | R | 1.71 |
| G | K | 1.62 |
| H | W | 1.55 |
| H | K | 1.46 |
| F | K | 1.45 |
| H | V | 1.42 |
| N | T | 1.39 |
| M | I | 1.36 |
| H | M | 1.17 |

### Example 8

### Measurement of Circulating FIX

The circulating half-life of FIX polypeptides is measured using an in vitro assay. This assay is based on the ability of FIX in vivo and in vitro to mediate the accumulation of adenovirus (Ad) in hepatocytes. Briefly, it has been shown that FIX can bind the Ad fiber knob domain and provide a bridge for virus uptake through cell surface heparin sulfate proteoglycans (HSPG) (Shayakhmetov, et al., J. Virol 79:7478-7491, 2005). An Adenovirus vector mutant, AdSmut, which contains mutations in the fiber knob domain, does not bind to FIX. AdSmut has significantly reduced ability to infect liver cells and liver toxicity in vivo, demonstrating that FIX plays a major role in targeting Ad vectors to hepatic cells (Shayakhmetov, et al., 2005). The ability of FIX to target Ad vector to hepatic cells can be blocked by inhibitors of protein-HSPG interactions (Shayakhmetov, et al., 2005).

Furthermore, HSPG-mediated uptake of FIX contributes significantly to FIX clearance and consequently, interfering with the HSPG interaction is expected to increase the half-life of FIX. Therefore, in vitro uptake of FIX and/or FIX variants in hepatocytes is measured, and variants with reduced uptake are expected to have increased half-life in vivo.

To measure FIX half-life in vitro, mammalian cells are incubated with adenovirus in the presence or absence of FIX or FIX variants. Viral uptake is mediated by wild-type FIX and measured by expression of the reporter gene encoded in viral genome, for example, green fluorescent protein (GFP) or luciferase expression. Reduced uptake of adenovirus in the presence of FIX variants are measured as reduced reporter gene expression, for example, reduced GFP fluorescence or reduced luciferase enzymatic activity as compared to wild-type FIX.

FIX circulating half-life is measured in vivo using standard techniques well-known to those of ordinary skill in the art. Briefly, the respective dose of FIX or FIX variant is administered to a subject by intravenous injection. Blood samples are taken at a number of time points after injection and the FIX concentration is determined by an appropriate assay (e.g., ELISA). To determine the half-life, that is the time at which the concentration of FIX is half of the concentration of FIX immediately after dosing, the FIX concentration at the various time points is compared to the FIX concentration expected or measured immediately after administering the dose of FIX. A correlation between reduced cellular uptake in the in vitro assay and increased half-life in the in vivo assay is expected.

### Example 9

### GlycoPEGylation of modified FIX

Approximately 5 mg of a modified FIX protein was buffer-exchanged into Reaction Buffer (25 mM HEPES, pH7.7, 50 mM NaCl, 10 mM CaCl₂, 0.01% TWEEN-80) to remove sucrose and amino acids which interfere with conjugation reactions, then loaded on to a HiTrap Desalting 5 ml column (Sephadex G25) with AKTA-FPLC chromatography system (GE) at a flow rate of 1 ml/min using a 1-ml sample loop (Reaction Buffer as mobile phase). Protein fractions were collected and pooled (˜2 ml) into a screw-cap tube. To this FIX solution (˜2.1 mg/ml), sodium meta-periodate (Sigma #311448, Mw213.89, NaIO₄) stock solution (400 mM aqueous solution, freshly made) was added to reach a final [NaIO₄] of 2 mM for mild oxidation, producing reactive aldehydes on the carbohydrate moieties of the FIX. The mixture was incubated at 4° C. for 60 minutes in the dark on a rotator. Sodium meta-periodate concentrations as low as 0.5 mM are also effective.

The oxidation step was then terminated by quenching residual NaIO₄ with 2M glycerol aqueous stock (to a final concentration of 20 mM glycerol) in an additional incubation of 15 minutes at 4° C. The oxidation reaction mixture (˜2 ml) was directly loaded onto the G25 column again as described above to separate the oxidized recombinant FIX from excess NaIO₄, glycerol and glyceraldehydes that would otherwise interfere with the subsequent PEGylation reaction.

To the resulting oxidized FIX solution in the Reaction Buffer (˜0.95 mg/ml, 4.3 mg in 4.5 ml), 80 mg Hydrazine-PEG30 (40× molar excess, NOF Catalog# SUNBRIGHT ME-300 HZ) and 10 mM aniline (1M stock solution in 100% EtOH) were added, and the PEGylation reaction was carried out overnight, on a rotating platform, at 4° C. The optimal condition for the PEGylation reaction was found to be 0.3 to 0.9 mg/ml [FIX] with added Hydrazine-PEG30 at 5- to 40-fold molar excess over [FIX]. PEGylation time can be further optimized to alter the ratio of mono-PEGylated FIX to di-PEGylated FIX.

Extensive characterization of GlycoPEG FIX by SDS-PAGE, Coomassie blue, iodine staining, Western blot analysis and Size-exclusion chromatography demonstrated that GlycoPEGylated FIX contained approximately 70% mono-PEGylated FIX and 30% di-PEGylated FIX. Further optimization of the glycoPEGylation method for FIX was achieved by reducing the sodium meta-periodate concentration to 0.5 mM, using a 5-fold molar excess of aminooxy-PEG at a Factor IX concentration of 0.6 mg/ml, optimizing the time of the PEGylation reaction, and purification on a heparin column followed by a size exclusion column. Using optimized conditions it was possible to achieve a 98.7% homogeneous PEGylated species. The rate and extent of carbohydrate oxidation by periodate can be controlled by reaction time, pH, temperature and concentration of periodate for example as described for antibodies by Wolfe and Hage, 1995 18. It has been reported that sialic acid residues on glycoproteins can be specifically oxidized with sodium periodate (NaIO4) by using 1 mM periodate and a temperature of 0° C. The site specificity of FIX glycopegylation could be optimized using 1 mM periodate or even lower concentrations. Optimization of quenching step might also be achieved. Finally the PEGylation step might be optimized for example by the use of PEG with different molecular weights, for example 5K, 10K, 15K, 20K, 30K, 40K, 60K or up to 150K. Alternative polymers might also be used as described above in the introduction. Alternative linker chemistry that utilizes alternative reactive groups attached to the PEG moiety or other polymer may also be used as described above in the introduction, for example including aminooxy PGE or Hydroxy-PEG-Amine.

### Example 10

### GlycoPEGylation of FIX-R338A using PEG-hydrazide

A BHK21 cell line expressing Human Factor IX containing the mutation R338A (FIX-R338A) was generated using standard methods and scaled up for fermentation in a 15 L scale perfusion reactor. The secreted FIX-R338A protein present in the media was purified to 98% purity by ion exchange chromatography. The resulting protein was subjected to glycoPEGylation using a 40 Kda PEG-Hydrazine as described above in the “Methods” section. The yield of PEGylated FIX-R338A could be increased from about 10% to about 50% by the inclusion of aniline as a catalyst during the PEGylation reaction. A large scale PEGylation on 5 mg of FIX-R338A was performed and the resulting protein was assayed for coagulation activity in vitro either by the aPTT assay (using elagic acid as the activator) or in a commercial chromagenic assay kit. Both assays used commercially produced recombinant wild type FIX (rFIX) to generate a standard curve. Controls of the starting material (FIX-R338A) and rFIX were run in each assay. The data shown in Table 5 indicated that the glycoPEGylated FIX-R338A had between 47% and 60% of the activity of the starting material but between 184% and 189% of the activity of rFIX.

TABLE 5
In vitro coagulation activity of GlycoPEGylated FIX-R338A

| | | Specific Activity |
|---|---|---|
| | Specific Activity | by chromagenic |
| Protein | by aPTT (IU/mg) | assay (IU/mg) |
| rFIX | 321 | 196 |
| FIX-R338A | 1270 | 621 |
| GlycoPEGylated FIX-R338A | 591 | 370 |
| GlycoPEGylated rFIX | not determined | 122 |

Specific activity as a percentage of un-PEGylated protein

| | | |
|---|---|---|
| GlycoPEGylated FIX-R338A % | 184% | 189% |
| of rFIX | | |
| GlycoPEGylated FIX-R338A % | 47% | 60% |
| of FIX-R338A | | |
| GlycoPEGylated rFIX % of rFIX | | 62% |

By combining PEGylation on sugars in the activation peptide with a higher activity variant of FIX it was possible to generate a PEGylated FIX with specific activity about 2-fold higher than that of recombinant wild type FIX protein. When rFIX was subjected to the same glycoPEGylation procedure and purification the resulting glycoPEGylated rFIX had a specific activity of 122 IU/mg by chromagenic assay which is 59% of the specific activity of un-modified rFIX. Thus compared to the glycoPEGylated recombinant wild type FIX, glycoPEGylated R338A had 3-fold higher specific activity which would enable 3-fold less protein to achieve the same therapeutic benefit.

Gel analysis of the glycoPEGylated FIX-R338A indicated that the protein contained a mixture of FIX-R338A PEGylated at only one site (mono-PEGylated) or a two sites (di-PEGylated). The Coomasie stained gel which stains proteins indicated the presence of two major PEGylated bands indicative of mono-PEGylated and di-PEGylated FIX-R338A. The Mono-PEGylated form appeared to be the predominate form.

### Example 11

### GlycoPEGylation of modified FIX using amino-oxy-PEG

Purified Factor IX (FIX) was first buffer-exchanged into Reaction Buffer (25 mM HEPES, pH 7.7, 50 mM NaCl, 10 mM CaCl₂, 0.01% w/v Tween-80) using a HiTrap Desalting 5 ml column (GE Healthcare) on an AKTA-FPLC chromatography system (GE Healthcare) at a flow rate of 1 ml/min. Protein fractions were collected and pooled. The FIX was oxidized by adding freshly prepared sodium meta-periodate (NaIO₄) (Sigma) from a 400 mM aqueous stock solution to a final concentration of 2 mM. Oxidation of FIX produces reactive aldehydes on the carbohydrate moieties of the FIX that can be modified by amino-oxy-PEG or hydrazine-PEG. The mixture was incubated at 4° C. for 60 minutes in the dark on a rotator. The NaIO₄ was quenched by the addition of 2M glycerol to a final concentration of 20 mM glycerol and further incubation for 15 minutes at 4° C. The oxidation reaction mixture was directly loaded onto the desalting column again as described above to separate the oxidized FIX from excess NaIO₄, glycerol and glyceraldehyde, which would interfere with subsequent PEGylation. To the resulting oxidized FIX solution (FIX concentration ˜0.5 mg/ml), a 40-fold molar excess of solid methoxy-PEG-30-oxyamine (NOF cat# SUNBRIGHT ME-300CA) and 10 mM aniline (1M stock solution in 100% EtOH) were added. The PEGylation reaction was carried out overnight, on a rotating platform, at 4° C. The optimal condition of the PEGylation reaction was found to be 0.3-0.9 mg/ml FIX with a 20-40-fold molar excess of PEG. Pegylation time can be further optimized to alter the ratio of resulting mono-PEGylated to di-PEGylated FIX.

The PEGylation reaction mixture was diluted 1:1 with Reaction Buffer and loaded onto a HiTrap™ Heparin HP 1-ml column (GE) using an AKTA chromatography system at 0.5 ml/min flow rate to purify PEGylated FIX. Free PEG did not bind to the heparin column. PEGylated FIX was separated from unpegylated FIX by gradient elution (0-100% Buffer B over 20-min). Buffer A was Reaction Buffer and Buffer B was 25 mM HEPES, pH 7.7, 500 mM NaCl, 20 mM CaCl₂, 0.01% w/v Tween-80). PEGylated FIX eluted first, followed by elution of the unPEGylated FIX. Fractions containing the PEGylated FIX were pooled and subjected to endotoxin removal. Possible endotoxin was removed using a 1-ml Endotrap column was packed with Profos® AG EndoTrap HD beads using pyrogen-free H2O. The column was attached to the AKTA system using sanitized tubing. The AKTA instrument, all lines, and the column were sanitized with 1N NaOH in 20% Ethanol for 1 hour followed by 0.1N Acetic Acid, 20% Ethanol for 2 hours. The column was then extensively washed with Milli-Q water. Regeneration Buffer (20 mM Tris-HCl pH7.5, 1M NaCl, 2 mM EDTA) was first applied to the column, then, the column was equilibrated with 50% Buffer B (25 mM HEPES, pH 7.7, 500 mM NaCl, 20 mM CaCl₂, 0.01% Tween-80) at 1 ml/min. PEGylated FIX from the heparin column was loaded onto the Endotrap column at 0.5 ml/min, and the flow through fraction, containing FIX, was collected into a sterile, pyrogen-free container.

Purified and endotoxin-free PEGylated FIX was concentrated, buffer-exchanged 6 times to Formulation buffer (0.234% NaCl, 8 mM histidine, 0.8% sucrose, 208 mM glycine, 0.004% Tween-80) by ultrafiltration (10K MW cutoff), aliquoted and stored at −80° C. after quick freezing. The protein concentration of GlycoPEG FIX was determined by measuring A₂₈₀ (extinction coefficient of 13.3 (mg/ml)⁻¹ cm⁻¹). Specific activity was calculated from the protein concentration and FIX chromogenic and aPTT assays (Ellagic acid activator). Possible contamination with FIXa and endotoxin was also evaluated by FIXa chromogenic and endotoxin detection assays. Additional biochemical characterization of GlycoPEG FIX was also performed (SDS-PAGE with Coomassie Blue and iodine staining, Western blot analysis, size-exclusion chromatography). demonstrated that GlycoPegylated FIX (Peak 1) contains 60% monoPEGylated FIX and 40% diPEGylated FIX. PEGylation efficiency was estimated at 50% and total recovery at 30%.

### Example 12

### GlycoPEGylation of FIX-R338A using PEG-amino-oxy

A BHK21 cell line expressing Human Factor IX containing the mutation R338A (FIX-R338A) was generated using standard methods and scaled up for fermentation in a 15 L scale perfusion reactor. The secreted FIX-R338A protein present in the media was purified to 98% purity by ion exchange chromatography. The resulting FIX-R338A protein was subjected to glycoPEGylation using a amino oxy-30 Kda PEG as described above. A PEGylation on 5 mg of FIX-R338A was performed and the resulting protein was assayed for coagulation activity in vitro either by the aPTT assay (using elagic acid as the activator) or in a commercial chromagenic assay kit. Both assays used commercially produced recombinant wild type FIX to generate a standard curve. Controls of the starting material (FIX-R338A) and rFIX were run in each assay. The data shown in Table 6 indicated that the glycoPEGylated FIX-R338A had between % and % of the activity of the starting material but between % and % of the activity of rFIX.

TABLE 6
In vitro coagulation activity of GlycoPEGylated FIX-R338A and
GlycoPEGylated rFIX generated using amino-oxy PEG

| | | Specific Activity |
|---|---|---|
| | Specific Activity | by chromagenic |
| Protein | by aPTT (IU/mg) | assay (IU/mg) |
| rFIX | 246 | 279 |
| FIX-R338A | 1623 | 1326 |
| GlycoPEGylated FIX-R338A | 661 | 970 |
| GlycoPEGylated rFIX | 120 | 198 |

Specific activity as a percentage

| | | |
|---|---|---|
| GlycoPEGylated FIX-R338A % | 41% | 73% |
| of FIX-R338A | | |
| GlycoPEGylated FIX-R338A % | 268% | 347% |
| of rFIX | | |
| GlycoPEGylated rFIX % of rFIX | 48% | 70% |
| GlycoPEGylated FIX-R338A % | 551% | 489% |
| of glycoPEGylated rFIX | | |

### Example 13

### GlycoPEGylation of Fix-R338A Using Peg-Amino-Oxy Under Conditions Optimized to Produce Homogeneous MonoPEGylated FIX-R338A

A BHK21 cell line expressing Human Factor IX containing the mutation R338A (FIX-R338A) was generated using standard methods and scaled up for fermentation in a 15 L scale perfusion reactor. The secreted FIX-R338A protein present in the media was purified to 98% purity by ion exchange chromatography. 10 mg of FIX-R338A protein was first buffer-exchanged into Reaction Buffer (25 mM HEPES, pH 7.7, 50 mM NaCl, 10 mM CaCl₂, 0.01% w/v Tween-80) using a HiTrap Desalting 5 ml column (GE Healthcare) on an AKTA-FPLC chromatography system (GE Healthcare) at a flow rate of 1 ml/min. Protein fractions were collected and pooled. The FIX was oxidized by adding freshly prepared sodium meta-periodate (NaIO₄) (Sigma) from a 400 mM aqueous stock solution to a final concentration of 0.5 mM. Oxidation of FIX produces reactive aldehydes on the carbohydrate moieties of the FIX that can be modified by amino-oxy-PEG. The mixture was incubated at 4° C. for 60 minutes in the dark on a rotator. The NaIO₄ was quenched by the addition of 2M glycerol to a final concentration of 20 mM glycerol and further incubation for 15 minutes at 4° C. The oxidation reaction mixture was directly loaded onto the desalting column again as described above to separate the oxidized FIX from excess NaIO₄, glycerol and glyceraldehyde, which would interfere with subsequent PEGylation. To the resulting oxidized FIX solution (FIX concentration ˜0.6 mg/me, a 5-fold molar excess over FIX protein of solid methoxy-PEG-30-oxyamine (NOF cat# SUNBRIGHT ME-300CA) and 10 mM aniline (1M stock solution in 100% EtOH) were added. The PEGylation reaction was carried out for 2 hours on a rotating platform, at 4° C. The PEGylation reaction mixture was diluted 1:1 with Reaction Buffer and loaded onto a HiTrap™ Heparin HP 1-ml column (GE) using an AKTA chromatography system at 0.5 ml/min flow rate to purify PEGylated FIX. Free PEG did not bind to the heparin column. PEGylated FIX was separated from unpegylated FIX by gradient elution (0-100% Buffer B over 20-min). Buffer A was Reaction Buffer and Buffer B was 25 mM HEPES, pH 7.7, 500 mM NaCl, 20 mM CaCl₂, 0.01% w/v Tween-80). PEGylated FIX eluted first, followed by elution of the unPEGylated FIX. Fractions containing mostly the mono-PEGylated FIX were pooled and subjected size exclusion chromatography (SD200) to further separate monoPEGylated FIX-R338A, diPEGylated FIX-R338A and free FIX-R338A. Fractions containing 95% homogenous monoPEGylated FIX were collected, concentrated and dialyzed back in to formulation buffer (0.234% NaCl, 8 mM histidine, 0.8% sucrose, 208 mM glycine, 0.004% Tween-80), aliquoted and stored at −80° C. after quick freezing. The protein concentration of GlycoPEG FIX-R338A was determined by measuring A₂₈₀ (extinction coefficient of 13.3 (mg/ml)⁻¹ cm⁻¹). Specific activity was calculated from the protein concentration and FIX chromogenic and aPTT assays (Ellagic acid activator). Both assays used commercially produced recombinant wild type FIX to generate the standard curve. Controls of the starting material (FIX-R338A). The data shown in Table 7 indicated that the glycoPEGylated FIX-R338A had between 34% and 80% of the activity of the starting material, depending on the assay.

TABLE 7
In vitro coagulation activity of 95% homogenous GlycoPEGylated
FIX-R338A generated using amino-oxy PEG and
optimized conditions

| | | Specific Activity |
|---|---|---|
| | Specific Activity | by chromagenic |
| Protein | by aPTT (IU/mg) | assay (IU/mg) |
| FIX-R338A | 1512 | 1174 |
| GlycoPEGylated FIX-R338A | 519 | 942 |

Specific activity as a percentage

| | | |
|---|---|---|
| GlycoPEGylated FIX- | 34% | 80% |
| R338A % of FIX-R338A | | |

### Example 14

### Pharmacokinetic Profile of glycoPEGylated FIX-R338A

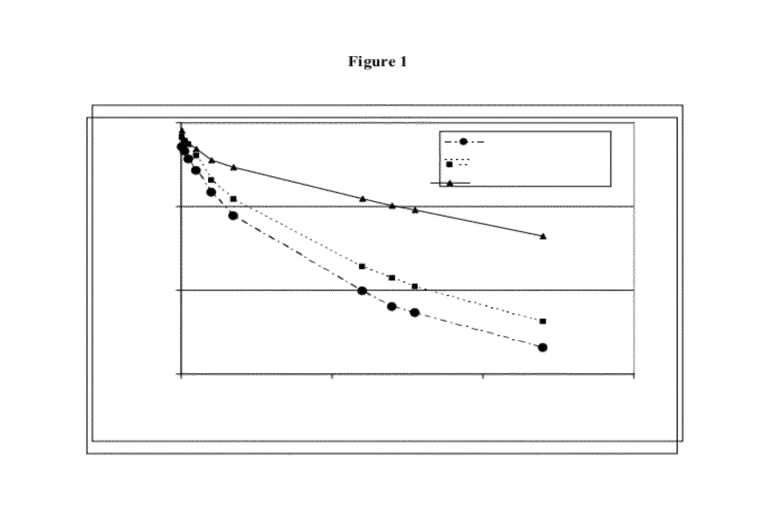

GlycoPEGylated FIX-R338A, FIX-R338A or recombinant wild type FIX (rFIX) were administered to normal rats or Hemophilia B mice by intravenous injection. The circulating level of FIX protein was measured over time using a ELISA based assay. In normal rats the pharmacokinetic profile of glycoPEGylated FIX-R338A was significantly improved as compared to both FIX-R338A and rFIX (FIG. 1).

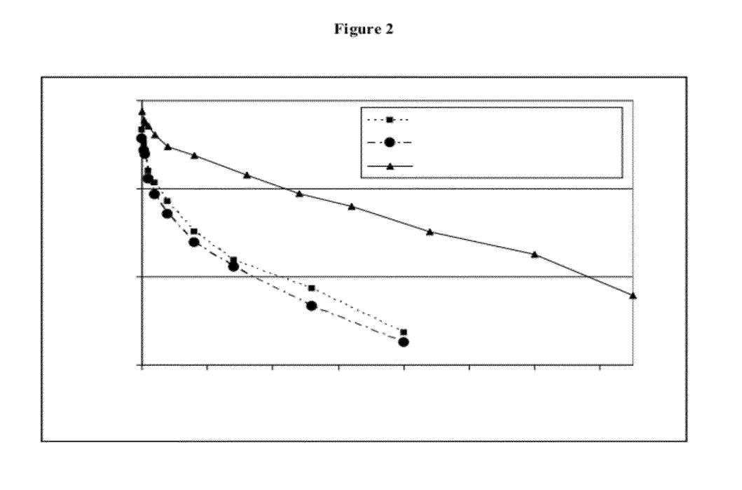

In Hemophilia B mice the pharmacokinetic profile of glycoPEGylated FIX-R338A was also significantly improved as compared to both FIX-R338A and rFIX (FIG. 2).

The pharmacokinetic parameters calculated from these studies (Tables 8 and 9) indicated that glycoPEGylated FIX-R338A had an improvement in the terminal half life (T½) of about 1.4-fold in rats and 1.5-fold in mice. The overall clearance was reduced by 3 to 4-fold in rats and by 6 to 8-fold in mice. Both dose normalized area under the curve (AUCnorm) and mean residence time (MRT) were also increased in both species.

TABLE 8
Pharmacokinetic parameters for glycoPEGylated
FIX-R338A in rats

| | T½ | CL (ml/ | Vss(ml/ | AUCnorm | MRT |
|---|---|---|---|---|---|
| Protein | (h) | h/kg) | kg) | (kg/h/l) | (h) |
| rFIX | 11 | 22 | 195 | 47 | 9 |
| FIX-R338A | 11 | 32 | 240 | 31 | 7.5 |
| GlycoPEGylated | 15 | 8 | 156 | 130 | 20.5 |
| FIX-R338A | | | | | |

TABLE 9
Pharmacokinetic parameters for glycoPEGylated
FIX-R338A in Hemophilia B mice

| | T 1/2 | CL (ml/ | Vss(ml/ | AUCnorm | MRT |
|---|---|---|---|---|---|
| Protein | (h) | h/kg) | kg) | (kg/h/l) | (h) |
| rFIX | 17.5 | 32.6 | 535 | 30.7 | 16.4 |
| FIX-R338A | 17.2 | 42.6 | 661 | 23.5 | 15.5 |
| GlycoPEGylated | 26.5 | 5.31 | 188 | 188 | 35.3 |
| FIX-R338A | | | | | |

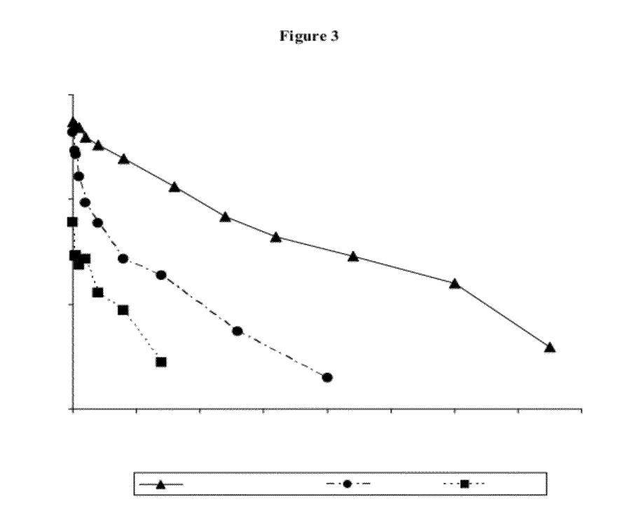

The FIX activity was also determined in plasma samples from the hemophilia B mice at different times after intravenous injection of either rFIX, FIX-R338A or glycoPEGylated FIX-R338A as shown in FIG. 3. These data demonstrate a significantly improved PK profile by activity for the PEGylated FIX-R338A molecule

### Example 15

### Aniline as a Catalyst for PEGylation of Factor IX

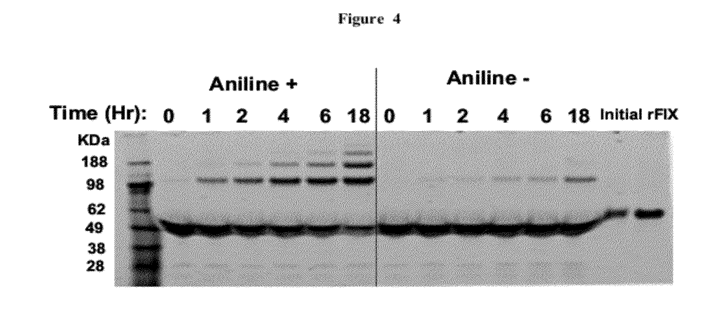

To evaluate aniline as a catalyst for conjugation of polymer moieties, such as PEG, to sugars on proteins, including FIX, recombinant WT-FIX protein was PEGylated as described in example 11 except that one reaction contained 10 mM aniline while a second identical reaction was performed without the addition of aniline The time course of the PEGylation reaction was monitored by analysis on SDS-PAGE (FIG. 4). In the presence of aniline the efficiency of PEGylation, as evidenced by the conversion of the 55 Kda free FIX protein to higher molecular weight forms, was increased. Quantitation of the gel indicated that after an 18 hr reaction only 18% of the free FIX was PEGylated in the absence of aniline while 73% of the free FIX was PEGylkated in the presence of aniline, demonstrating that aniline improved the rate of PEG conjugation.

### Example 16

### Site Specific Polymer Conjugation on Sugars of Factor IX by Mutation at Either N157 or N167

Factor IX contains two N-linked glycosylation sites located at N157 and N167 and the glycans that are added at these sites during protein expression in mammalian cells contain the majority of the silaic acid moieties present on the total glycans of Factor IX. Conjugation of polymers such as PEG to the sialic acids of Factor IX as described in examples 9 to 15 may occur on either or both of the glycans attached to N157 and N167. It would be desirable from a pharmaceutical perspective to produce a polymer conjugated Factor IX in which the polymer is attached at only one of the two N-lunked glycosylation sites because such a product would be more homogenous. Factor IX containing the R338A mutation was mutated to change either N157 to A157 or N167 to A167, thus ablating each of the N-linked glycosylation sites. N157Q and N167Q are predicted to be alternate mutations to ablate the respective N-linked glycosylation sites due to the structural similarity between the asparagine (N) and glutamine (Q) residues. Expression of R338A-N157A and R338A-N167A in BHK21 cells and measurement of the antigen level by ELISA and the activity by aPTT assay in the cell culture supernatants demonstrated that the N167A mutein had similar specific activity (expressed as IU per mg of FIX protein) to that of the parental FIX-R338A protein (Table 10). In contrast, the N157A mutein exhibited a 1.7-fold higher specific activity than the parental FIX-R338A protein (Table 10). A similar 1.7 fold higher specific activity was measured for the purified FIX-R338A-N157A protein as compared to the FIX-R338A protein (Table 10). Therefore mutation of N157 such as N157A or N157Q to remove the N-linked glycosylation site at N157 and thus enabling polymer conjugation preferentially at N167 are preferred over mutations at N167 for the purpose of generating a homogenous polymer conjugated Factor IX protein.

TABLE 10
Specific activity of N157A and N167A muteins in cell
culture supernatants and purified proteins

| Protein | | Specific Activity | Fold |
|---|---|---|---|
| source | FIX protein | (IU/mg) | R338A |
| Cell culture | FIX-R338A | 319 | — |
| supernatants | FIX-R338A-N167A | 320 | 0.99 |
| | FIX-R338A-N157A | 529 | 1.66 |
| Purified | FIX-R338A | 1297 | — |
| Protein | FIX-R338A-N167A | NT | — |
| | FIX-R338A-N157A | 2230 | 1.72 |

(NT: not tested)

All publications and patents mentioned in the above specification are incorporated herein by reference. Various modifications and variations of the described methods of the invention will be apparent to those skilled in the art without departing from the scope and spirit of the invention.

Although the invention has been described in connection with specific embodiments, it should be understood that the invention as claimed should not be unduly limited to such specific embodiments. Indeed, various modifications of the above-described modes for carrying out the invention which are obvious to those skilled in the field of biochemistry or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A Factor IX polypeptide comprising an amino acid sequence that has been modified by introducing one or more amino acid substitutions.

2. The polypeptide of claim 1, wherein said polypeptide comprises an amino acid substitution at residue 86.

3. The polypeptide of claim 1, wherein said polypeptide comprises one or more amino acid substitutions selected from amino acid residues 85, 86, and 87.

4. The polypeptide of claim 1, wherein said polypeptide comprises one or more amino acid substitutions selected from amino acid residues 85, 86, 87, 338, and 410.

5. The polypeptide of claim 4, wherein the one or more amino acid substitutions are selected from D85F; D85G; D85H; D851; D85M; D85N; D85R; D85S; D85W; D85Y; V86A; V86D; V86E; V86G; V86H; V861; V86L; V86M; V86N; V86P; V86Q; V86R; V86S; V86T; T87F; T871; T87K; T87M; T87R; T87V; T87W; R338A; R338F; R3381; R338L; R338M; R338S; R338T; R338V; R338W; E410N; E410Q; D85W and T87R; D85F and T871; D85W and T87W; D85R and T85R; D851 and T87R; D85Y and T87F; D851 and T87M; D85F and T87R; D85F and T87V; D85R and T87K; D85H and T871; D851 and T871; D85Y and T87K; D85S and T87R; D85Y and T87R; D85G and T87K; D85H and T87W; D85H and T87K; D85F and T87K; D85H and T87V; D85M and T871; D85H and T87M; R338A and E410N; R338A and E410Q; D85W, V86A, and T87R; D85F, V86A, and T871; D85W, V86A, and T87W; D85R, V86A, and T85R; D851, V86A, and T87R; D85Y, V86A, and T87F; D851, V86A, and T87M; D85F, V86A, and T87R; D85F, V86A, and T87V; D85R, V86A, and T87K; D85H, V86A, and T871; D851, V86A, and T871; D85Y, V86A, and T87K; D85S, V86A, and T87R; D85Y, V86A, and T87R; D85G, V86A, and T87K; D85H, V86A, and T87W; D85H, V86A, and T87K; D85F, V86A, and T87K; D85H, V86A, and T87V; D85M, V86A, and T871; D85H, V86A, and T87M; D85W, V86A, T87R, and R338A; D85F, V86A, T871, and R338A; D85W, V86A, T87W, and R338A; D85R, V86A, T85R, and R338A; D851, V86A, T87R, and R338A; D85Y, V86A, T87F, and R338A; D851, V86A, T87M, and R338A; D85F, V86A, T87R, and R338A; D85F, V86A, T87V, and R338A; D85R, V86A, T87K, and R338A; D85H, V86A, T871, and R338A; D851, V86A, T871, and R338A; D85Y, V86A, T87K, and R338A; D85S, V86A, T87R, and R338A; D85Y, V86A, T87R, and R338A; D85G, V86A, T87K, and R338A; D85H, V86A, T87W, and R338A; D85H, V86A, T87K, and R338A; D85F, V86A, T87K, and R338A; D85H, V86A, T87V, and R338A; D85M, V86A, T871, and R338A; D85H, V86A, T87M, and R338A; D85W, V86A, T87R, R338A, and E410N; D85F, V86A, T871, R338A, and E410N; D85W, V86A, T87W, R338A, and E410N; D85R, V86A, T85R, R338A, and E410N; D851, V86A, T87R, R338A, and E410N; D85Y, V86A, T87F, R338A, and E410N; D85I, V86A, T87M, R338A, and E410N; D85F, V86A, T87R, R338A, and E410N; D85F, V86A, T87V, R338A, and E410N; D85R, V86A, T87K, R338A, and E410N; D85H, V86A, T87I, R338A, and E410N; D85I, V86A, T87I, R338A, and E410N; D85Y, V86A, T87K, R338A, and E410N; D85S, V86A, T87R, R338A, and E410N; D85Y, V86A, T87R, R338A, and E410N; D85G, V86A, T87K, R338A, and E410N; D85H, V86A, T87W, R338A, and E410N; D85H, V86A, T87K, R338A, and E410N; D85F, V86A, T87K, R338A, and E410N; D85H, V86A, T87V, R338A, and E410N; D85M, V86A, T87I, R338A, and E410N; D85H, V86A, T87M, R338A, and E410N; D85W, V86A, T87R, R338A, and E410Q; D85F, V86A, T87I, R338A, and E410Q; D85W, V86A, T87W, R338A, and E410Q; D85R, V86A, T85R, R338A, and E410Q; D85I, V86A, T87R, R338A, and E410Q; D85Y, V86A, T87F, R338A, and E410Q; D85I, V86A, T87M, R338A, and E410Q; D85F, V86A, T87R, R338A, and E410Q; D85F, V86A, T87V, R338A, and E410Q; D85R, V86A, T87K, R338A, and E410Q; D85H, V86A, T87I, R338A, and E410Q; D85I, V86A, T87I, R338A, and E410Q; D85Y, V86A, T87K, R338A, and E410Q; D85S, V86A, T87R, R338A, and E410Q; D85Y, V86A, T87R, R338A, and E410Q; D85G, V86A, T87K, R338A, and E410Q; D85H, V86A, T87W, R338A, and E410Q; D85H, V86A, T87K, R338A, and E410Q; D85F, V86A, T87K, R338A, and E410Q; D85H, V86A, T87V, R338A, and E410Q; D85M, V86A, T87I, R338A, and E410Q; D85H, V86A, T87M, R338A, and E410Q; and any combination thereof.

6. A Factor IX polypeptide comprising the amino acid sequence
(SEQ ID NO: 2)
YNSGKLEEFVQGNLERECMEEKCSFEEAREVFENTERTTEFWKQYVDGD
QCESNPCLNGGSCKDDINSYECWCPFGFEGKNCELX₈₅X₈₆X₈₇CNIKN
GRCEQFCKNSADNKVVCSCTEGYRLAENQKSCEPAVPFPCGRVSVSQTS
KLTRAETVFPDVDYVNSTEAETILDNITQSTQSFNDFTRVVGGEDAKPG
QFPWQVVLNGKVDAFCGGSIVNEKWIVTAAHCVETGVKITVVAGEHNIE
ETEHTEQKRNVIRIIPHHNYNAAINKYNHDIALLELDEPLVLNSYVTPI
CIADKEYTNIFLKFGSGYVSGWGRVFHKGRSALVLQYLRVPLVDRATCL
X₃₃₈STKFTIYNNMFCAGFHEGGRDSCQGDSGGPHVTEVEGTSFLTGII
SWGEECAMKGKYGIYTKVSRYVNWIKX₄₁₀KTKLT;
wherein X₈₅ is selected from D, F, G, H, I, M, N, R, S, W, and Y;
wherein X₈₆ is selected from A, D, E, G, H, I, L, M, N, P, Q, R, S, T, and V;
wherein X₈₇ is selected from F, I, K, M, R, T, V, and W;
wherein X₃₃₈ is selected from A, F, I, L, M, R, S, T, V, and W;
wherein X₄₁₀ is selected from E, N, and Q.

7. The polypeptide of any of claims 1 to 6, further comprising one or more glycosylation sites.

8. A pharmaceutical preparation comprising the Factor IX polypeptide of any one of claims 1-7 and a pharmaceutically acceptable carrier.

9. A method of treating hemophilia B comprising administering to a subject in need thereof a therapeutically effective amount of the pharmaceutical preparation of claim 8.

10. A DNA sequence encoding the polypeptide of any one of claims 1-7.

11. A eukaryotic host cell transfected with the DNA sequence according to claim 10 in a manner allowing the host cell to express a Factor IX polypeptide.

12. A method for producing a Factor IX polypeptide comprising (i) modifying the amino acid sequence of the polypeptide by introducing one or more amino acid substitutions; (ii) expressing the polypeptide in a cell line; and (iii) purifying the polypeptide.

13. The polypeptide of claim 7, further comprising one or more sugar moieties attached to said one or more glycosylation sites.

14. The polypeptide of claim 13, wherein the one or more sugar moiety is a sialic acid.

14. A conjugate comprising a) the polypeptide of claim 13 or 14, and b) one or more polymer moieties covalently attached thereto.

15. The conjugate of claim 14, wherein the one or more polymer moieties is covalently attached to one or more sugar moieties.

16. The conjugate of claim 15, wherein the one or more polymer moiety is selected from the group consisting of a poly(alkylene glycols), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(alpha-hydroxy acid), poly(vinyl alcohol), polyphosphazene, polyoxazoline, poly(N-acryloylmorpholine), polysialic acid, hydroxyethyl starch (HES), polyethylene oxide, alkyl-polyethylene oxides, bispolyethylene oxides, co-polymers or block co-polymers of polyalkyene oxides, poly(ethylene glycol-co-propylene glycol), poly(N-2-(hydroxyproply)methyacrylamide), and dextran.

17. The conjugate of claim 16, wherein the one or more polymer moiety is a poly(alkylene glycol).

18. The conjugate of claim 17, wherein the poly(alkylene glycol) is polyethylene glycol (PEG).

19. A conjugate comprising:
a) a Factor IX polypeptide comprising an amino acid sequence that has been modified by introducing one or more amino acid substitutions, wherein at least one amino acid substitution is at residue 338;
b) one or more sugar moieties attached to said one or more glycosylation sites; and
c) one or more polymer moieties covalently attached to one or more sugar moieties.

20. The conjugate of claim 19, wherein said substitution at residue 338 is selected from the group consisting of R338A, R338F, R338I, R338L, R338M, R338S, R338T, R338V, and R338W.

21. The conjugate of claim 19 or 20, wherein said polypeptide further comprises one or more amino acid substitutions selected from amino acid residues 157 and 167.

22. The conjugate of claim 21, wherein said substitution at residue 157 is selected from the group consisting of N157A and N157Q.

23. The conjugate of claim 21, wherein said substitution at residue 167 is selected from the group consisting of N167A and N167Q.

24. A method for improving conjugation of a polymer moiety to a polypeptide comprising: a) providing a polypeptide having one or more glycosylation sites, wherein the glycosylation site comprises one or more sialic acids; b) oxidizing said sialic acids of said polypeptide; c) providing a catalyst; and d) covalently attaching a polymer moiety comprising an amino-oxy functional group to said oxidized sialic acids; whereby the rate of conjugation is increased.

25. The method of claim 24, wherein said catalyst is selected from the group consisting of aniline and aniline derivatives such as o-Cl-, p-Cl-, o-CH3O-, p-CH3O-, and p-CH3-aniline.

26. The method of claim 24, wherein said rate of conjugation is increased relative to without the catalyst.
